(19) United States
(12) Patent Application Publication
(10) Pub. No.: US 2024/0290417 A1
(43) Pub. Date: Aug. 29, 2024

(54) SYSTEMS AND METHODS FOR GENE NETWORK INFERENCE

(71) Applicants: Steve Presse, Scottsdale, AZ (US); Max Schweiger, Phoenix, AZ (US); Camile Moyer, Oakland, CA (US); Zeliha Kilic, Memphis, TN (US)

(72) Inventors: Steve Presse, Scottsdale, AZ (US); Max Schweiger, Phoenix, AZ (US); Camile Moyer, Oakland, CA (US); Zeliha Kilic, Memphis, TN (US)

(73) Assignee: Arizona Board of Regents on Behalf of Arizona State University, Tempe, AZ (US)

(21) Appl. No.: 18/435,773

(22) Filed: Feb. 7, 2024

Related U.S. Application Data
(60) Provisional application No. 63/483,578, filed on Feb. 7, 2023.

(51) Int. Cl. G16B 5/20; G06N 5/04; G06N 7/01

(52) U.S. Cl. CPC G16B 5/20; G06N 5/04; G06N 7/01

(57) ABSTRACT

A system and associated method learns full distributions over gene states, state connectivities, and associated rate parameters, simultaneously and self-consistently from single molecule level RNA counts within a Bayesian nonparametric paradigm. The method propagates noise originating from fluctuating RNA counts over networks warranted by the data by treating networks themselves as random variables. The method is demonstrated on the lacZ pathway in Escherichia coli cells, the STL1 pathway in Saccharomyces cerevisiae yeast cells, and robustness is verified on synthetic data.

### CROSS-REFERENCE TO RELATED APPLICATIONS

This is a U.S. Non-Provisional Patent Application that claims benefit to U.S. Provisional Patent Application Ser. No. 63/483,578 filed 7 Feb. 2023, which is herein incorporated by reference in its entirety.

### GOVERNMENT SUPPORT

This invention was made with government support under RO1 GM130745 and RO1 GM134426 awarded by the National Institutes of Health. The government has certain rights in the invention.

### FIELD

The present disclosure generally relates to bioinformatics and transcriptomics, and in particular, to a system and associated method for gene state inference through Bayesian methods.

### BACKGROUND

Quantitative measurements of RNA dynamics in populations of fixed cells and individual living cells have consistently revealed complex distributions of RNA counts and behavior across space, time, and individual cells, even for clonal cell populations. Broadly, the term “gene expression variability” is invoked to explain these ubiquitous, variable, and complex RNA expression distributions. One of single-cell biology's driving goals is understanding the molecular origin and downstream consequences of gene expression variability. For example, recent work has demonstrated that rare cells, identifiable only by transient fluctuations in RNA content compared to clonal sister cells, can drive drug-resistant cancer or maintain progenitor cells driving development. While experimental methods can identify rare cells, robustly determining gene transcriptional states and their connectivities from discrete RNA counts across cells remains an open problem.

It is with these observations in mind, among others, that various aspects of the present disclosure were conceived and developed.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIG. 1A is an illustration showing various one, two, and three state gene models;

FIG. 1B is a simplified diagram showing a system for gene network inference outlined herein;

FIG. 2 is a simplified diagram showing an example computing device for implementation of various systems and methods described herein;

FIGS. 3A and 3B are a pair of process flow diagrams showing a computer-implemented method for gene network inference corresponding with the system of FIG. 1B;

FIG. 4 is a graphical representation showing a transcription rate sensitivity analysis including posterior probability distributions over production rates (βₗ) and transition rates (k_{σ}_{ₗ}_{→σ′}_{ₗ}) obtained by the system of FIG. 1B applying the method in FIGS. 3A and 3B, where each column shows comparable breadth of distributions for a network with two gene states under various maximum ground truth production rates (note that the posterior maximum closely matches the ground truth);

FIG. 5 is a graphical representation showing comparison of successful inference of degradation rates (γ) including posterior probability distributions over production rates (βₗ) and transition rates (k_{σ}_{ₗ}_{→σ}_{ₗ}) obtained by the system of FIG. 1B applying the method in FIGS. 3A and 3B, where the left-hand column shows inference of degradation rates (γ) and the right-hand column shows fixing of degradation rates (γ) for identical data from a three gene state model;

FIG. 6 is a graphical representation showing posterior distributions over: gene states (first column), production rates (second column), degradation rates (third column), and transition rates (fourth column), where the first row shows distributions for a one gene state model, i.e., production, degradation, and no transition rates;

FIG. 7 is a graphical representation showing posterior distributions over production rates and transition rates, for networks with two gene states, where each row shows a different kinetic rate's posterior becoming increasingly narrow as the quantity of data used in the analysis grows;

FIGS. 8A-8C are a series of graphical representations showing results of analysis of the lacZ pathway in E. coli grown in glycerol at 30° C.; each panel represents the assigned posterior probability distribution for different model parameters shown as vertical cyan lines, pink shaded regions depict intervals in which 95% of MCMC samples lie, and the bottom panel shows log likelihood compared with other methods;

FIG. 9 is a graphical representation showing predictive distributions corresponding to FIGS. 8A-8C, resulting from simulation of 500 Gillespie trajectories per time point, demonstrating that even when parameters differ widely the system of FIG. 1B can predict nearly identical RNA distributions;

FIG. 10 is a graphical representation showing a subset of inferred rates for the lacZ pathway in E. coli grown in Glucose at 37° C., showing rates of production and transition associated to the states with the two greatest production rates;

FIG. 11 is a graphical representation showing parametric inference on E. coli data made by the system of FIG. 1B, specifically showing comparison between predicted rates when the model is constrained to only making predictions for a two gene state model with one nontranscribing state;

FIGS. 12A and 12B show the subset of inferred rates corresponding to linear switching between the four inferred gene states; and

FIG. 13 is a graphical representation showing predictive distributions corresponding to FIGS. 12A and 12B, resulting from simulation of 500 Gillespie trajectories per time point.

Corresponding reference characters indicate corresponding elements among the view of the drawings. The headings used in the figures do not limit the scope of the claims.

### SUMMARY

A system outlined herein for gene network inference includes a processor in communication with a memory, the memory including instructions executable by the processor to: access observation data associated with a gene network including a quantity of RNA for a plurality of cells observed across a plurality of time points; sample a set of probability values associated with observing the observation data for values of each respective parameter of a plurality of parameters through a measurement model (the plurality of parameters including: a set of discrete parameters including one or more gene states observable within the observation data and a success probability of each respective gene state of the one or more gene states being active; and a set of continuous parameters including kinetic rates associated with transitions between the one or more gene states based on a local geometry of the measurement model across the plurality of time points) and jointly infer, based on the set of probability values and the observation data, a set of most probable values of the plurality of parameters.

The memory can include instructions executable by the processor to apply a Gibbs sampling scheme to iteratively sample probability values associated with values of each respective model parameter of the gene network from the measurement model. Further, the memory can include instructions executable by the processor to: sample the success probability for a gene state of the one or more gene states using an adaptive Metropolis-Hastings sampling scheme encapsulated within the Gibbs sampling scheme; iteratively sample probability values associated with the set of continuous parameters using a Hamiltonian Monte Carlo sampling scheme encapsulated within the Gibbs sampling scheme; and apply a Parallel Tempering scheme nested within the Gibbs sampling scheme for iteratively sampling probability values associated with values of each respective model parameter of the gene network from the measurement model.

The measurement model can incorporate a nonparametric Bayesian formulation of a Chemical Master Equation that characterizes probabilistic temporal evolution of the gene network. Further, the nonparametric Bayesian formulation of the Chemical Master Equation can incorporate a load vector that dynamically represents activity or inactivity of the one or more gene states observable within the observation data. The measurement model can also include a joint posterior probability distribution expressive of a probability of observing the observation data given values of the plurality of parameters of the measurement model. The posterior probability distribution can be constructed based on a set of prior probability distributions associated with each respective parameter of the plurality of parameters of the measurement model. Each gene state of the one or more gene states can respectively be expressed as an element of a load vector, wherein a prior probability distribution associated with the load vector includes a Beta-Bernoulli process prior probability distribution and where a value of the element of the load vector representing activity or inactivity of the associated gene state.

In a further aspect, a method for gene network inference includes: accessing observation data associated with a gene network including a quantity of RNA for a plurality of cells observed across a plurality of time points; sampling a set of probability values associated with observing the observation data for values of each respective parameter of a plurality of parameters through a measurement model (where the plurality of parameters include a set of discrete parameters including one or more gene states observable within the observation data and a success probability of each respective gene state of the one or more gene states being active and a set of continuous parameters including kinetic rates associated with transitions between the one or more gene states based on a local geometry of the measurement model across the plurality of time points); and jointly inferring, based on the set of probability values and the observation data, a set of most probable values of the plurality of parameters.

The method can further include: applying a Gibbs sampling scheme to iteratively sample probability values associated with values of each respective model parameter of the gene network from the measurement model; applying a Parallel Tempering scheme nested within the Gibbs sampling scheme for iteratively sampling probability values associated with values of each respective model parameter of the gene network from the measurement model; sampling the success probability for a gene state of the one or more gene states using an adaptive Metropolis-Hastings sampling scheme encapsulated within the Gibbs sampling scheme; and iteratively sampling probability values associated with the set of continuous parameters using a Hamiltonian Monte Carlo sampling scheme encapsulated within the Gibbs sampling scheme.

The measurement model can incorporate a nonparametric Bayesian formulation of a Chemical Master Equation that characterizes probabilistic temporal evolution of the gene network. Further, the nonparametric Bayesian formulation of the Chemical Master Equation can incorporate a load vector that dynamically represents activity or inactivity of the one or more gene states observable within the observation data. The measurement model can further include a joint posterior probability distribution expressive of a probability of observing the observation data given values of the plurality of parameters of the measurement model, where each gene state of the one or more gene states being respectively expressed as an element of a load vector, and where a prior probability distribution associated with the load vector includes a Beta-Bernoulli process prior probability distribution.

### DETAILED DESCRIPTION

### 1. Introduction

Accessing information on what drives a biological process often involves interrupting the process under observation and collecting snapshot data reporting back on the underlying network to be inferred. As measurement snapshot data themselves are stochastic, they necessarily motivate the development of probabilistic inference schemes to deduce the underlying reaction networks such as, for example, gene states and their connectivities from RNA counts. In such networks, nodes represent gene states, and edges represent biochemical reaction rates linking states. Simultaneously estimating reaction networks' constituent parameters and quantifying their uncertainties from snapshot data remains a challenging task.

Attempts to infer states and their connectivities under these conditions face three immediate challenges: 1) inherently stochastic measurements introduce uncertainty over which reaction network models may be warranted by the data; and 2) the number of allowed nodes, their connecting edges, and values for rates may be unknown; and 3) rates parameterizing these networks may be separated by multiple orders of magnitude. While Bayesian nonparametric methods rigorously propagate measurement uncertainty into uncertainty over states and their connectivities alongside parameters, resolving challenge (1), appropriately sampling these posteriors given items (2) and (3) remains an open question. The present disclosure outlines systems and methods that apply a hybrid Bayesian Markov Chain Monte Carlo (MCMC) sampler, directly addressing challenges (2) and (3) leveraging three methods: Hamiltonian Monte Carlo (HMC) which leverages local posterior geometries in inference to explore the parameter space effectively; Adaptive Metropolis Hastings (AMH) which learns correlations between plausible parameter sets to efficiently propose probable models; and Parallel Tempering which takes into account multiple models simultaneously with tempered information content. The methods are applied to data from single molecule Fluorescence in-situ Hybridization (FISH), a popular snapshot method probing transcriptional networks to illustrate the identified challenges and how the methods outlined herein address them.

Gene expression models (e.g., gene states and their connectivities), key toward understanding a cell's regulatory response, underlie experimental observations of single cell transcriptional dynamics. While RNA expression data encodes information on gene expression models, existing computational frameworks do not perform simultaneous Bayesian inference of gene expression models and parameters from such data. Rather, gene networks (composed of gene states, their connectivities, and associated parameters including kinetic parameters) are currently deduced by pre-specifying gene state numbers and connectivity prior to learning associated rate parameters. As such, the correctness of gene networks cannot be independently assessed which can lead to strong biases. By contrast, the present disclosure outlines systems and methods that enable simultaneously and self-consistently learning full distributions over gene states, state connectivities, and associated rate parameters from single molecule level RNA counts. Notably, the methods propagate noise originating from fluctuating RNA counts over gene networks warranted by the data by treating gene networks themselves as random variables, achieved by operating within a Bayesian nonparametric paradigm. The method is demonstrated on the lacZ pathway in Escherichia coli cells, the STL1 pathway in Saccharomyces cerevisiae yeast cells, and verify its robustness on synthetic data.

### 2. Challenges

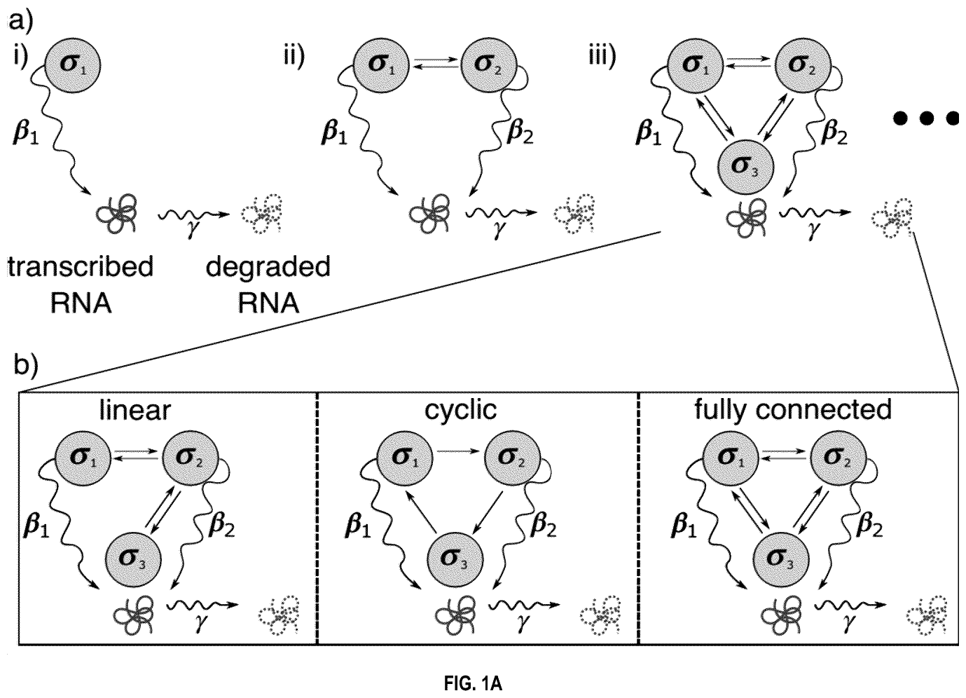

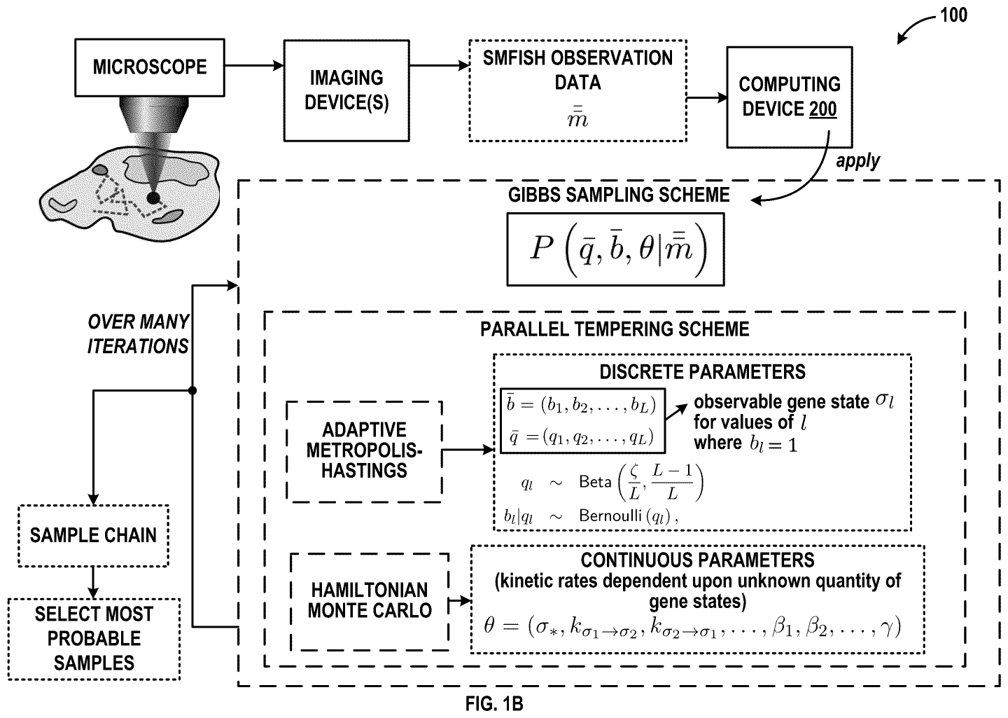

FIG. 1A shows examples of gene networks defined by their number of gene states, state connectivities, and associated rate parameters. Each grey circle depicts an RNA production state that a gene may occupy, differentiated by its unique production rate. Straight arrows reflect possible transitions between gene states, and curved arrows depict RNA transcription (with rate β) or degradation (with rate γ). FIG. 1A also depicts models with a variety of transitions omitted.

Toward unraveling gene expression models, the present disclosure considers experimental methods providing discrete RNA counts, focusing on single-molecule RNA Fluorescence in situ Hybridization (smFISH). In particular, smFISH provides snapshot data (referred to herein as “observation data”, see FIG. 1B) that includes independent fluorescent imaging assays performed on fixed samples at discrete time points, often following external stimuli. These assays yield the number and location of individual transcripts for a limited number of RNA species for individual cells and, as a consequence, direct insight into the molecular state of the cells or tissue at the time of fixation.

To help highlight the major challenges facing computational inference of gene networks from snapshot smFISH data, consider a simple gene network, shown at the top left corner of FIG. 1A, which includes a single gene state. The one-state model predicts a Poissonian number of RNAs transcribed per time interval. However, for some genes, a Poisson statistical expectation may disagree with observations, and, as such, the perennial two-state model (top middle of FIG. 1A) is conjectured. The two-state, or telegraph, model allows the gene expression to transition between an inactive and active state. Though the two-state model often provides reasonable agreement with data, higher-state models may provide even better agreement. This, in turn, suggests the possibility that gene states with intermediate RNA production rates may exist. Immediately, challenges emerge on account of the many ways of connecting N≥3 gene states (lower portion of FIG. 1A for some examples). The additional reaction pathways may provide a better fit to existing data at the cost of predictive power. In fact, model inference, which rigorously balances data description with predictive power, has yet to be achieved for this problem. In a number of previous attempts, metrics (including Poisson indicators, cross-validation, nonparametric regression, or information metrics which compare a truncated set of possible models) are used to justify the introduction and network structure of additional gene states (top right corner of FIG. 1A). However, all such methods perform model selection and parameter inference separately and cannot therefore propagate error from inherently stochastic RNA counts into uncertainty over gene networks. As such, they fail to balance descriptive ability and predictive power in a statistically rigorous manner, and the relative probability over each proposed network, including gene states and associated parameters and thus connectivity of the gene states, given the data remains unknown.

In a further simplification, some of these methods altogether ignore the intrinsic stochasticity of RNA counts in favor of mass action formulations, which predict the temporal evolution of the mean number of RNA molecules per cell. Mass action formulations are fundamentally insufficient for smFISH data, as RNA copies may be present at low numbers, rendering information on their copy number fluctuations vital toward extracting kinetic parameters. Even methods which resolve this issue using the forward Kolmogorov equation (chemical master equation or CME), which gives the probabilistic temporal evolution of single-cell RNA counts, estimate the number of gene states without rigorous Bayesian uncertainty propagation.

Many previous methods, particularly those advertised as less costly than Bayesian inference, fall into the broad category of Maximum Likelihood Estimation (MLE) methods. Some of these, in particular Adaptive Differential Evolution, may agree with Bayesian methods of the kind explored further herein. However, any algorithm of choice in obtaining MLEs would return a unique model, even if the data does not warrant such strong conclusions.

Furthermore, any attempt to determine which unique gene expression model is warranted by the data, would require integrating the (necessarily approximated) CME for many different sets of parameters in MLE estimation, with the added cost of estimating parameters across models separately. Therefore not only are methods to pick out unique models and their MLE parameters costly, but they also do not propagate inherent uncertainty from stochastic RNA counts into uncertainty over models. What is more, they do not leverage the computational benefit that may be derived from exploring models in parameter estimation. Thus, moving beyond MLE, the present disclosure is motivated by methods that simultaneously and self-consistently learn gene expression models and parameters from data.

Based on these observations, the present disclosure outlines a Bayesian framework evaluated using Markov Chain Monte Carlo (MCMC) to simultaneously derive and sample from a probability over gene states, their associated parameters (and thus gene state connectivity), given discrete RNA counts taken from smFISH snapshot data, The resulting Bayesian method outlined herein propagates inherent noise into model (e.g., gene state number) estimation.

This can be achieved within the Bayesian paradigm, which allows sampling from posterior probability distributions over gene networks. In order to construct these posteriors, prior probability distributions over gene state numbers are required, necessitating the use of Bayesian Nonparametrics (BNPs) which overcomes the problem of “overfitting” in model determination by placing priors over infinite candidate models. As with “normal” (parametric) Bayesian methods, a likelihood incorproates the data in a way that requires an approximate solution of the CME. With a likelihood and nonparametric priors at hand, the systems and methods outlined herein are shown to simultaneously and self-consistently estimate model structures (number of gene states) alongside associated rate parameters (and thus, gene state connectivity) between states, as warranted by observed stochastic RNA counts per cell.

Applicability of the methods are demonstrated by testing on synthetic data followed by two very different experimental systems: the IacZ pathway in Escherichia coli (E. coli) cells and the STL1 pathway in Saccharomyces cerevisiae (S. cerevisiae) yeast cells.

### 3. Methods

### 3.1 Model Formulation

FIG. 1B shows a general overview of a system 100 for gene network inference using smFISH observation data, which can be in the form of RNA counts per cell, mₜ_{ₖ}^{j}, collected at time points t_{1:K}, and cells indexed as j=1, . . . , Jₖ. For simplicity, RNA counts from all cells at all time points are denoted as

$${m}^{=} = {\{ {\{ {m}_{{t}_{k}}^{j} \}}_{j = 1 : {J}_{k}} \}}_{k = 1 : K} .$$

Using this information, the goal is to infer the gene expression model, i.e., infer both gene states and their associated rate parameters (and thus gene state connectivity).

In each gene state, indexed σₗ, the gene transcribes RNA copies at rate βₗ. All RNA degrade stochastically according to overall rate γ. A gene can transition stochastically, say from state σₗ to σ_{l′}, at rate k_{σ}_{ₗ}_{→σ′}_{ₗ}. For convenience, all parameters are collectively recruited under the symbol:

$$θ = ( {σ}_{*} , {k}_{{σ}_{1} \to {σ}_{2}} , {k}_{{σ}_{2} \to {σ}_{1}} , … , {β}_{1} , {β}_{2} , … , γ )$$

where σₗ=σ denotes the initial gene state.

In order to infer θ within the Bayesian paradigm, a likelihood, P(m̅|θ) must be specified first. Given measurements m̅, the likelihood is given by:

$$P ( {m}^{=} \vert θ ) = {\prod }_{k = 1}^{K} {\prod }_{j = 1}^{{J}_{k}} ( {\sum }_{l = 1}^{N} {P}_{θ}^{{t}_{k}} ( {σ}_{l} , {m}_{{t}_{k}}^{j} ) )$$

with P_{θ}^{τ}≡(P_{θ}^{τ}(σₗ1), . . . , P_{θ}^{τ}(σₗ, M), P_{θ}^{τ}(σ₂, 1), . . . , P_{θ}^{τ}(σ₂, M), . . . , P_{θ}^{τ}(σ_{N}1), . . . , P_{θ}^{τ}(σ_{L}M))T satisfying the CME, \dot{P}_{θ}^{τ}=A·P_{θ}^{τ}, where A is a generator matrix, whose dependency on θ is outlined in more detail in Section 5.2.

### 3.2 Model Inference

In order to construct a posterior using the likelihood, prior probability distributions (priors) are required over all model parameters. The priors over quantities in θ can be selected for computational convenience and are detailed in Section 5.4.

This section expands upon the nonparametric prior used on gene states.

A nonparametric formulation must theoretically consider an infinite number of gene states and allow the data to winnow down these infinite possibilities to those warranted by the data. This is similar to regular (parametric) Bayesian methods which typically assume broad priors over parameters and eventually allow the data, incorporated through the likelihood, to sharpen parameter estimates (i.e., sharpen the posterior).

As a matter of computational convenience, the Beta-Bernoulli process is selected as a formal prior on the existence or non-existence of these states.

Put simply, an infinite number of intermediate binary (Bernoulli) indicator variables, bₗ, termed loads, are introduced. Each respective load equals 1 when an associated gene state σₗ is deemed necessary by the data, or 0 otherwise. To make computation feasible, a so-called weak limit L is introduced that sets an upper bound on the number of possible gene states. A load vector that collects loads {bₗ}l=1: L is referred to collectively as b̅, (where each possible gene state is represented by an element of the load vector).

The Beta-Bernoulli process prior on the loads reads:

$${q}_{l} \sim Beta ( \frac{ζ}{L} , \frac{L - 1}{L} ) \\ ( {b}_{l} \vert {q}_{l} ) \sim Bernoulli ( {q}_{l} ) ,$$

where qₗ are hyperparameters describing the success probability of load bₗ being “active” or equal to 1, and ζ is a hyperhyperparameter. Given this prior, one can learn from the data which gene states are warranted (e.g., by determining which elements of the load vector can be assigned values of “1”).

Given the likelihood and all priors, an explicit form can now be constructed for the posterior probability distribution, P(q̅, b̅, θ|m̅). As the likelihood does not assume an analytic form, the system (e.g., system 100 shown in FIG. 1B) can generate pseudo-random numbers from P(q̅, b̅, θ|m̅) using a custom Markov Chain Monte Carlo (MCMC) sampling scheme.

Importantly, the ability to efficiently explore this posterior, especially given the added difficulty of inferring states, enables escape from traps (local maxima) that have impacted the assessment of parameters of other methods (Section 6.2.2).

With this fact in mind, the system 100 can use an overall Gibbs sampling scheme to construct the Markov Chain of the MCMC sampling scheme. Within this Gibbs sampling scheme, the system can sample the initial condition, σ_{*,} and loads, b̅, directly from their joint marginal posterior distribution. By contrast, the system samples success probabilities q using a Metropolis-Hastings sampling scheme. Because: 1) the model is simultaneously learning discrete (number of gene states, initial condition), and continuous (kinetic rates) parameters; and 2) there is a significant scale separation between various individual continuous parameters, featureless posterior distributions may be encountered over large portions of the possible model space.

To address problem 1), all parameters are sampled with Parallel Tempering (PT) in order to better explore the discrete parameters. Within the PT scheme, continuous parameters are proposed using Hamiltonian Monte Carlo (HMC) sampling, solving problem 2). Used in conjunction for the first time, these sampling schemes permit the inference of gene states and their associated parameters on reasonable time scales, avoiding local maxima mentioned earlier in Section 6.2.1.

### 4. Computer-Implemented System and Method

### 4.1 Computing Device

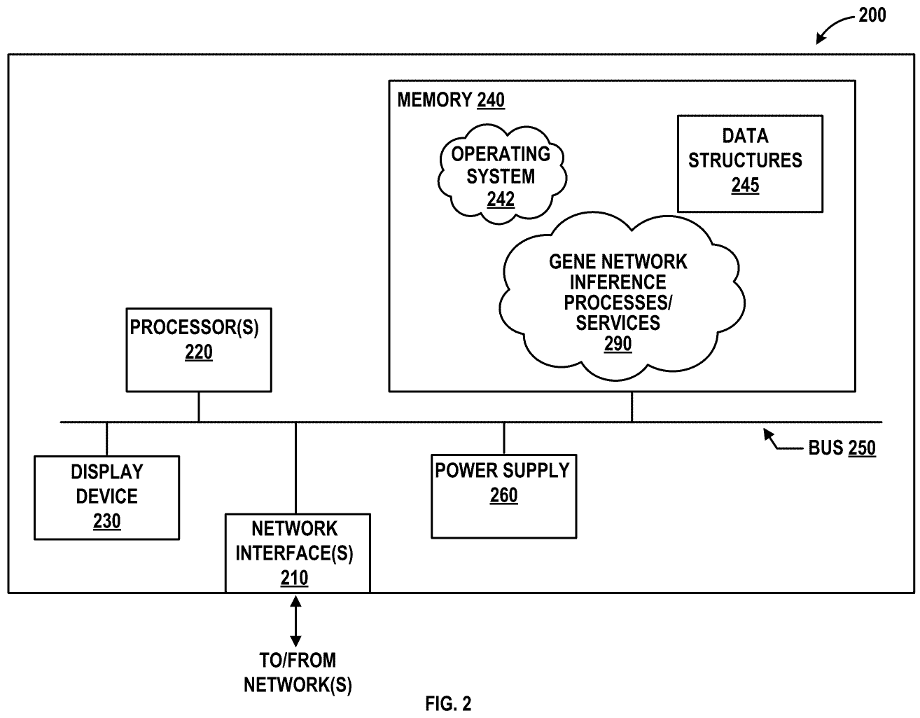

FIG. 2 is a schematic block diagram of an example device 200 that may be used with one or more embodiments described herein, e.g., as a component of the system 100 of FIG. 1B and associated methods outlined herein.

Device 200 comprises one or more network interfaces 210 (e.g., wired, wireless, PLC, etc.), at least one processor 220, and a memory 240 interconnected by a system bus 250, as well as a power supply 260 (e.g., battery, plug-in, etc.). Device 200 may also include or otherwise communicate with a display device 230 which can communicate the results (identified parameters and gene states) to a user based on the smFISH observation data.

Network interface(s) 210 include the mechanical, electrical, and signaling circuitry for communicating data over the communication links coupled to a communication network. Network interfaces 210 are configured to transmit and/or receive data using a variety of different communication protocols. As illustrated, the box representing network interfaces 210 is shown for simplicity, and it is appreciated that such interfaces may represent different types of network connections such as wireless and wired (physical) connections. Network interfaces 210 are shown separately from power supply 260, however it is appreciated that the interfaces that support PLC protocols may communicate through power supply 260 and/or may be an integral component coupled to power supply 260.

Memory 240 includes a plurality of storage locations that are addressable by processor 220 and network interfaces 210 for storing software programs and data structures associated with the embodiments described herein. In some embodiments, device 200 may have limited memory or no memory (e.g., no memory for storage other than for programs/processes operating on the device and associated caches). Memory 240 can include instructions executable by the processor 220 that, when executed by the processor 220, cause the processor 220 to implement aspects of the various systems and methods outlined herein.

Processor 220 comprises hardware elements or logic adapted to execute the software programs (e.g., instructions) and manipulate data structures 245. An operating system 242, portions of which are typically resident in memory 240 and executed by the processor, functionally organizes device 200 by, inter alia, invoking operations in support of software processes and/or services executing on the device. These software processes and/or services may include gene network inference processes/services 290, which can include aspects of methods and/or implementations of various modules described herein. Note that while gene network inference processes/services 290 is illustrated in centralized memory 240, alternative embodiments provide for the process to be operated within the network interfaces 210, such as a component of a MAC layer, and/or as part of a distributed computing network environment.

It will be apparent to those skilled in the art that other processor and memory types, including various computer-readable media, may be used to store and execute program instructions pertaining to the techniques described herein. Also, while the description illustrates various processes, it is expressly contemplated that various processes may be embodied as modules or engines configured to operate in accordance with the techniques herein (e.g., according to the functionality of a similar process). In this context, the term module and engine may be interchangeable. In general, the term module or engine refers to model or an organization of interrelated software components/functions. Further, while the gene network inference processes/services 290 is shown as a standalone process, those skilled in the art will appreciate that this process may be executed as a routine or module within other processes.

### 4.2 Computer-Implemented Method

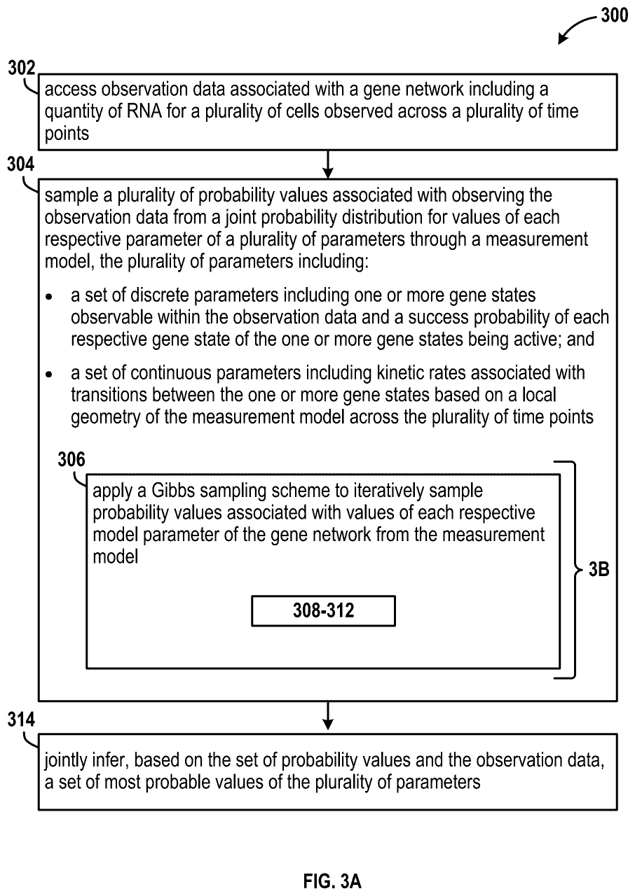

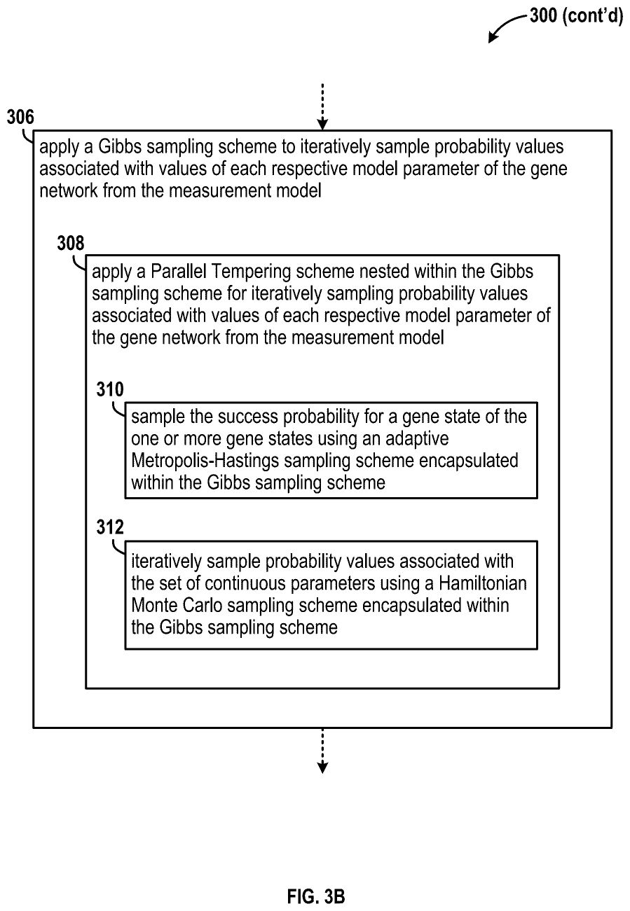

A method 300 outlined herein and shown in FIGS. 3A and 3B for gene network inference may be implemented using device 200 (e.g., as part of gene network inference processes/services 290) in accordance with the system 100 shown in FIG. 1B. The method 300 corresponds with FIG. 1A and its corresponding discussion, as well as the Inverse Model presented in section 6 of the present disclosure.

Referring to FIG. 3A, step 302 of method 300 includes accessing observation data associated with a gene network including a quantity of RNA for a plurality of cells observed across a plurality of time points. In the examples outlined herein, observation data (in the form of smFISH data) is denoted as observed RNA count mi across a set of cells for the plurality of time points.

Step 304 of method 300 includes sampling a plurality of probability values from a joint probability distribution associated with observing the observation data for values of each respective parameter of a plurality of parameters through a measurement model. The plurality of parameters include: a set of discrete parameters including one or more gene states observable within the observation data and a success probability of each respective gene state of the one or more gene states being active; and a set of continuous parameters including kinetic rates associated with transitions between the one or more gene states based on a local geometry of the measurement model across the plurality of time points. The measurement model can incorporate a nonparametric Bayesian formulation of a Chemical Master Equation that characterizes probabilistic temporal evolution of the gene network. The nonparametric Bayesian formulation of the Chemical Master Equation can incorporate a load vector that dynamically represents activity or inactivity of the one or more gene states observable within the observation data.

Step 304 can be achieved through step 306, which can include applying a Gibbs sampling scheme to iteratively sample probability values associated with values of each respective model parameter of the gene network from the measurement model.

Referring to FIG. 3B, in order to more effectively explore discrete parameter values, each iteration of the Gibbs sampling scheme of step 306 can include step 308. Step 308 includes applying a Parallel Tempering scheme nested within the Gibbs sampling scheme for iteratively sampling probability values associated with values of each respective model parameter of the gene network from the measurement model.

Steps 310 and 312 can be nested within the Parallel Tempering scheme of step 308, which is nested within each iteration of the Gibbs sampling scheme of step 306. Step 310 applies to discrete parameters (gene states, number of gene states, etc.) and includes sampling the success probability for a gene state of the one or more gene states using an adaptive Metropolis-Hastings sampling scheme encapsulated within the Gibbs sampling scheme. Step 312 applies to continuous parameters (kinetic rates) and includes iteratively sampling probability values associated with the set of continuous parameters using a Hamiltonian Monte Carlo sampling scheme encapsulated within the Gibbs sampling scheme.

Referring back to FIG. 3B, after conclusion of steps 304-312, step 314 includes jointly inferring, based on the set of probability values and the observation data, a set of most probable values of the plurality of parameters.

### 5. Model Derivation and Computational Strategy

### 5.1 Model Overview

This section outlines and describes the computational structure including formulation of the measurement model and construction of the inference strategy. The method operates on observation data in the form of single cell RNA count data, with mₜ_{ₖ}^{j} representing a single cell RNA count for cell j taken at time point tₖ for k=1, 2, . . . , K and j=1, . . . , Jₖ, collected as m̅={m̅ₜ_{ₖ}}ₖ₌₁^{K}, where m={mₜ_{ₖ}^{j}}ⱼ₌₁^{J}. Given these single cell RNA counts, information can be extracted about the underlying gene state model driving the observed dynamics. The gene state model includes kinetic parameters including transition rates between gene states (and thus gene state connectivity), production rates, and degradation rates.

To infer these kinetic parameters, a gene state model is presented. The defining characteristic of the model, the number of gene states, ultimately dictates the number of kinetic parameters involved in the underlying dynamics.

Below, the present disclosure first describes a simpler analog of the method for deriving kinetic parameter approximations when the number of gene states is held at a fixed quantity, i.e., a parametric framework. Then, the present disclosure expands the inference framework to the nonparametric paradigm to infer both the number of gene states and kinetic parameters.

### 5.2 Model Description

Working within a Bayesian paradigm, the given single cell RNA counts must first be connected to the model parameters. For ease of explanation, the present disclosure starts by describing a parametric formulation, for which the number of gene states is provisionally fixed to two. For the two-state model, each gene state can be labeled using σₗ for l=1, 2. Now, all associated kinetic parameters can be described which include the initial gene state σ_{*,} either σ₁ or σ₂; the transition rates, k_{σ}_{₁}_{→σ}_{₂} and k_{σ}_{₂}_{→σ}_{₁}; the production rates, β₁ and β₂; and the degradation rate, γ, all grouped into θ=(σ_{*}, k_{σ}_{₁}_{→σ}_{₂}, k_{σ}_{₂}_{Γσ}_{₁}, β₁, β₂, γ).

Armed with θ, the kinetic parameters are related to the single cell RNA counts m̅ with the measurement model. This measurement model employs the chemical master equation (CME). More specifically, the solution to the CME can be used, P_{θ}^{t}(σ₁, m), which gives the probability of encountering m RNA in a cell residing in gene state σ₁ at time t given model parameters θ. Dynamics of these probabilities are governed by a generator matrix, denoted by A, whose structure is dictated by the underlying gene state model.

### 5.2.1 Generator Matrix

Here, A is a square block matrix which depends on θ and whose size reflects the number of gene states.

To describe the structure of A, the present disclosure starts with the nonzero elements associated with the nth row, arbitrarily chosen, for which n<M and is therefore associated to the derivative dP_{θ}^{t}(σ₁, n)/dt. The nonzero elements include:

$$A ( n , 1 : 2 * ( M + 1 ) ) = \{ \begin{matrix}A ( n , n - 1 ) \\ A ( n , n ) \\ A ( n , n + 1 ) \\ A ( M + 1 + n , n - 1\end{matrix} \} = \{ \begin{matrix}{β}_{1} \\ - ( {k}_{{σ}_{1} \to {σ}_{2}} + {β}_{1} + n γ ) \\ ( n + 1 ) γ \\ {k}_{{σ}_{1} \to {σ}_{2}}\end{matrix} \} .$$

Note that, as the number of RNA is unbounded, the infinite set of CME ordinary differential equations (ODEs) can be truncated by a preset maximum (M) RNA count within a given dataset.

Given the generator matrix's form, A, the CME's structure is defined for which the solution, P_{θ}^{t}(σ₁, m), satisfies:

$$\frac{d}{dt} P ( t \vert θ ) = A \cdot P ( t \vert θ ) \\ where : \\ P ( t \vert θ ) = {( {P}_{θ}^{t} ( {σ}_{1} , 0 ) , {P}_{θ}^{t} ( {σ}_{1} , 1 ) , … , {P}_{θ}^{t} ( {σ}_{1} , M ) , \\ {P}_{θ}^{t} ( {σ}_{2} , 0 ) , {P}_{θ}^{t} ( {σ}_{2} , 2 ) , … , {P}_{θ}^{t} ( {σ}_{2} , M ) )}^{T}$$

Provided the above, the likelihood is constructed, P(\dot{m}|θ), which is a measure of how probable the data, m is given a set of parameters θ. Since the single cell RNA counts are independent and identically distributed, a likelihood of the following form is built:

$$P ( {m}^{=} \vert θ ) = {\prod }_{k = 1}^{K} {\prod }_{j = 1}^{{J}_{k}} ( {\sum }_{l = 1}^{N} {P}_{θ}^{{t}_{k}} ( {σ}_{l} , {m}_{{t}_{k}}^{j} ) ) . \qquad ( 2 )$$

Likelihoods for the one state and three state models follow a similar structure and are described in detail below.

Moving into the nonparametric regime, the likelihood must be adjusted dynamically based upon the number of gene states introduced into the gene state model during model inference. Therefore, intermediate binary indicator variables are introduced, bₗ, termed as loads, which correspond to each possible gene state and can be either 1 or 0. The loads affect the configuration of A, and thus the CME's solution, by multiplying all rates associated to their respective gene state. Specifically, the production rate for gene state n, βₗ, is multiplied by load bₗ. Similarly, the transition rate from gene state l to gene state l′, k_{σ}_{ₗ}_{→σ′}_{ₗ}, is multiplied by loads bₗ and b′ₗ as both gene states must exist for a transition between them to be relevant.

Finally, the degradation rate is multiplied by each respective load, since if a gene state is inactive, the gene will never occupy that state, and therefore degradation will not occur in that state. This change is demonstrated using the same nonzero elements of A as were outlined for the parametric two state model,

$$A ( n , 1 : L * ( M + 1 ) ) = \{ \begin{matrix}A ( n , n - 1 ) \\ A ( n , n ) \\ A ( n , n + 1 ) \\ A ( M + 1 + n , n - 1\end{matrix} \} = \{ \begin{matrix}{b}_{1} , {β}_{1} \\ - ( {b}_{1} {\sum }_{l = 2}^{L} {b}_{l} {k}_{{σ}_{1} \to {σ}_{l}} + {b}_{1} {β}_{1} + {nb}_{1} γ ) \\ ( n + 1 ) {b}_{1} γ \\ {b}_{1} {\sum }_{l = 2}^{L} {k}_{{σ}_{l} \to {σ}_{1}}\end{matrix} \} .$$

where L is chosen as a weak limit imposed on the maximum possible number of gene states for computational feasibility. In this case, this row corresponds to the derivative dP_{b,θ}^{t} (σ₁, n)/dt which is as described for the two state model but now incorporates the new dependencies on the load variables, grouped as b=(b₁, b₂, . . . , b_{L}), as shown for A (n, 1:L*(M+1)).

In this way, each load determines whether or not its corresponding gene state, and thus the associated rates, is warranted by the data. The model parameters encompassed in θ grow to include all possible transition rates and production rates.

### 5.3 Model Inference

In the Bayesian paradigm, now that the likelihood is defined, priors must be placed over all parameters to be inferred. Choices for priors over the kinetic parameters as well as the initial condition gene state are relatively straightforward and are described in Section 5.4. To place priors over the loads, bₙ, the system can employ Beta-Bernoulli process priors:

$${q}_{l} \sim Beta ( \frac{ζ}{L} , \frac{L - 1}{L} ) \\ ( {b}_{l} \vert {q}_{l} ) \sim Bernoulli ( {q}_{l} ) ,$$

where qₙ re hyperparameters which describe the success probability of load bₙ being “active” or equal to 1, and ζ is a hyperhyperparameter. Given this setup, it is possible to determine which gene states are deemed necessary by the data.

Given the likelihood and all priors, the fully joint posterior probability distribution can be defined for both parametric, P(θ|m̅) and nonparametric, P((q̅, b̅, θ|m̅)), frameworks. As there is no possible fully conjugate prior (over all variables) associated to the likelihood, which does not have a closed form solution due to the CME, the posterior probability also does not attain a closed form. Therefore, the system can employ a Markov Chain Monte Carlo (MCMC) scheme which will enable generation of pseudo-random samples from the posteriors.

The main bottleneck in the model is the high computational expense associated with numerically approximating CME solutions. In particular, as the size of the CME is given by the structure of the generator matrix, A, the computational cost grows for data sets associated with high RNA counts (highly expressed genes) and gene state models incorporating higher numbers of gene states.

### 5.4 Summary of Equations

Loads are defined as: b̅=(b₁, b₂, . . . , b_{L}); transition rates are defined as: k̅=(k_{σ}_{ₗ}_{→σ′}_{ₗ}) for l=1, . . . , L, l′=1, . . . , L and l≠l; and production rates are defined as: β̅=(β₁, β₂, . . . , β_{L}). The success probability vector, q̅=(q₁, q₂, . . . , q_{L}) and the initial condition gene state σ.

\tilde{β}ₗ=log₁₀(βₗ) and a similar convention is kept for all other rates. Finally, the set of parameters are collected in \tilde{θ}=(\overline{\tilde{k}}, \tilde{β̅},\tilde{γ}). The full set of equations employed by the system now follows:

$${q}_{l} \sim Beta ( \frac{ζ}{L} , \frac{L - 1}{L} ) , l = 1 , … , L$$

$${σ}_{*} \vert \overline{q} \sim Categorical ( \frac{\overline{q}}{{\sum }_{l = 1}^{L} {q}_{l}} )$$

$${b}_{l} \vert {q}_{l} , {σ}_{*} \sim Bernoulli ( {δ}_{{σ}_{*} l} + ( 1 - {δ}_{{σ}_{*} l} ) {q}_{l} ) , l = 1 , … , L$$

$${\tilde{k}}_{{σ}_{l} \to {σ}_{{l}^{′}}} \sim Normal ( {ϕ}_{{1}_{{ll}^{′}}} , {ψ}_{{1}_{{ll}^{′}}} ) , l = 1 , … , L , {l}^{′} = 1 , … , J , l \neq {l}^{′}$$

$${\tilde{β}}_{l} \sim Normal ( {ϕ}_{{2}_{l}} , {ψ}_{{2}_{l}} ) , l = 1 , … , L ,$$

$$\tilde{γ} \sim Normal ( {ϕ}_{3} , {ψ}_{3} )$$

$${m}^{=} \vert {σ}_{*} , \overline{b} , θ \sim {\prod }_{k = 1}^{K} {\prod }_{j = 1}^{{J}_{k}} ( {\sum }_{i = 1}^{L} {P}^{t , {σ}_{*} , \overline{b} , θ} ( {σ}_{i} , {m}_{k}^{j} ) ) .$$

### 5.5 Nonparametric Chemical Master Equation

Let P^{t, σ, b̅, θ} (σₗ, m) be the probability of a cell being in gene state σₗ with m RNA count, m, at time t given an initial gene state of σ_{*} and parameters θ, such that:

$${P}^{t , {σ}_{*} , \overline{b} , θ} ( {σ}_{l} , m ) = ( {P}^{t , {σ}_{*} , \overline{b} , θ} ( {σ}_{l} , 0 ) , {P}^{t , {σ}_{*} , \overline{b} , θ} ( {σ}_{l} , 1 ) , … {P}^{t , {σ}_{*} , \overline{b} , θ} ( {σ}_{l} , M ) )$$

where M is the maximum number of RNA per cell considered (and fixed at some high albeit reasonable number for computational efficiency). Then the master equation for the BNP model, in matrix form, reads:

$$\frac{d}{dt} \begin{matrix}{P}^{t , {σ}_{*} , \overline{b} , θ} ( {σ}_{1} , m ) \\ {P}^{t , {σ}_{*} , \overline{b} , θ} ( {σ}_{1} , m ) \\ ⋮ \\ {P}^{t , {σ}_{*} , \overline{b} , θ} ( {σ}_{L} , m )\end{matrix} ] = [ \begin{matrix}{T}_{1} & {K}_{{σ}_{2} \to {σ}_{1}} & … & {K}_{{σ}_{L} \to {σ}_{1}} \\ {K}_{{σ}_{1} \to {σ}_{2}} & {T}_{2} & ⋱ & {K}_{{σ}_{L} \to {σ}_{2}} \\ ⋮ & ⋱ & ⋱ & ⋮ \\ {K}_{{σ}_{1} \to {σ}_{L}} & {K}_{{σ}_{2} \to {σ}_{L}} & … & {T}_{L}\end{matrix} ] [ \begin{matrix}{P}^{t , {σ}_{*} , \overline{b} , θ} ( {σ}_{1} , m ) \\ {P}^{t , {σ}_{*} , \overline{b} , θ} ( {σ}_{2} , m ) \\ ⋮ \\ {P}^{t , {σ}_{*} , \overline{b} , θ} ( {σ}_{L} , m )\end{matrix}$$

with the matrix,

$${T}_{l} = ( \begin{matrix}- ( {b}_{l} {β}_{l} + {\sum }_{j = 1}^{L} {b}_{l} {b}_{{l}^{′}} {k}_{{σ}_{l} \to {σ}_{{l}^{′}}} ) & {b}_{l} γ & 0 & 0 & … \\ {b}_{l} {β}_{l} & - ( {b}_{l} {β}_{l} + {\sum }_{{l}^{′} = 1}^{L} {b}_{l} {b}_{{l}^{′}} {k}_{{σ}_{l} \to {σ}_{{l}^{′}}} + {b}_{l} γ ) & 2 {b}_{l} γ & 0 & ⋱ \\ 0 & {b}_{l} {β}_{l} & - ( {b}_{l} {β}_{l} + {\sum }_{j = 1}^{L} {b}_{l} {b}_{{l}^{′}} {k}_{{σ}_{l} \to {σ}_{{l}^{′}}} + 2 {b}_{l} γ ) & 3 {b}_{l} γ & ⋱ \\ ⋮ & ⋱ & ⋱ & ⋱ & ⋱ \\ 0 & 0 & … & {b}_{l} β - l & - ( {b}_{l} {β}_{l} + {\sum }_{{l}^{′} = 1}^{L} {b}_{l} {b}_{{l}^{′}} {k}_{{σ}_{l} \to {σ}_{l}^{′}} + {Mb}_{l} γ )\end{matrix} )$$

provided that k_{σ}_{ₗ}_{→σ′}_{ₗ}=0 for l=1, . . . , L, and K_{σ}_{ₗ}_{→σ′}_{ₗ}, are diagonal matrices with k_{σ}_{ₗ}_{→σ′}_{ₗ}, along the diagonals for l=1, . . . L and l′=1, . . . L.

Initial condition used is:

$${P}^{0 , {σ}_{*} , \overline{b} , θ} ( {σ}_{l} , 0 ) = 1 , for l = {σ}_{*} and m = 0 \\ {P}^{0 , {σ}_{*} , \overline{b} , θ} ( {σ}_{l} , 0 ) = 0 , otherwise$$

### 5.6 Description of the Computational Scheme

In some examples, the system can employ an overall Gibbs sampling scheme to construct the Markov Chain. The Gibbs scheme is a method for updating a full set of parameters which completely parameterize a model. For convenience, the present disclosure denotes this complete set of parameters as Λ. In each step of the Gibbs scheme, the system obtains a new set of parameters, Λ, from a set of old parameters Λ^{Old} by proposing multiple disjoint subsets θᵢ^{prop} of Λ for i=1: I, which together, would completely parameterize the model. For each i in sequence, the system applying the Gibbs scheme provisionally updates the full set of parameters to Λ^{prop}, using the values which make up θᵢ^{prop}. Then, the system compares the posterior probability of Λ^{prop} (which now contains the parameters from θᵢ^{prop} in addition to the most recent remaining parameters in Λ) is compared to that of Λ. If the comparison favors Λ^{prop}, then the update Λ=Λ^{prop} is made, otherwise Λ remains unchanged. In this way, once all I subsets have been assessed and either accepted or rejected, the system can obtain new estimates for all parameters in Λ.

Within the Gibbs sampling scheme, the system samples the initial conditions σ_{*} and b̅ directly from their joint marginal posterior distribution (Section 5.6.1). By contrast, the system samples success probabilities q̅ using a Metropolis-Hastings sampling scheme (Section 5.6.2). Because: 1) the system is simultaneously learning discrete (number of gene states, initial condition), and continuous (kinetic rates) parameters; and 2) there is a significant scale separation between various individual continuous parameters; the system may encounter featureless posterior distributions over large portions of the possible model space. To address problem 1), the system samples all parameters with Parallel Tempering (PT) in order to better explore the discrete parameters. Within the PT scheme, the system proposes all continuous parameters (kinetic rates) using Hamiltonian Monte Carlo (HMC) (Section 5.6.6) sampling, solving problem 2).

### 5.6.1 Joint Sampling of the Loads and Initial Condition Gene State

The system can use direct sampling within a Gibbs sampling scheme to sample the initial condition, σ_{*} and bₗ for l=1, . . . , L, jointly from the marginal posterior distribution:

$$\begin{matrix}\begin{matrix}ℙ ( {σ}_{*} , \overline{b} \vert {m}^{=} , \overline{q} , θ ) ∝ \\ ℙ ( {m}^{=} \vert {σ}_{*} , \overline{b} , θ ) ℙ ( \overline{b} \vert \overline{q} , {σ}_{*} ) ℙ ( {σ}_{*} \vert \overline{q} )\end{matrix} = {\prod }_{k = 1}^{K} {\prod }_{j = 1}^{{J}_{k}} ( {\sum }_{l = 1}^{L} {P}^{t , {σ}_{*} , \overline{b} , θ} ( {σ}_{l} , {m}_{k}^{j} ) ) \\ {\prod }_{l}^{L} Bernoulli ( {b}_{l} ; {δ}_{{σ}_{*} l} + \\ ( 1 - {δ}_{{σ}_{*} l} ) {q}_{l} ) \times \\ Categorical ( {σ}_{*} ; \frac{\overline{q}}{{\sum }_{l = 1}^{L} {q}_{l}} ) \\ = {\prod }_{k = 1}^{K} {\prod }_{j = 1}^{{J}_{k}} ( {\sum }_{l = 1}^{L} {P}^{t , {σ}_{*} , \overline{b} , θ} ( {σ}_{l} , {m}_{k}^{j} ) ) \times \\ {\prod }_{l}^{L} {( {δ}_{{σ}_{*} l} + ( 1 - {δ}_{{σ}_{*} l} ) {q}_{l} )}^{{b}_{l}} ( {δ}_{{σ}_{*} l} + \\ {( 1 - {δ}_{{σ}_{*} l} ) {q}_{l} )}^{1 - {b}_{l}} ( \frac{{q}_{{σ}_{*}}}{{\sum }_{l = 1}^{L} {q}_{l}} )\end{matrix} .$$

The system can sample initial condition gene state and the loads through posteriors of all possible initial condition gene states with all possible load combinations:

$$\begin{matrix}{P}_{1} = ℙ ( {C}_{1} \vert {m}^{=} , {\overline{ϕ}}_{*} , θ ) , & {C}_{1} = ( {σ}_{*} = 1 , \overline{b} = [ 1 , 0 , … , 0 ] ) \\ {P}_{2} = ℙ ( {C}_{2} \vert {m}^{=} , {\overline{ϕ}}_{*} , θ ) , & {C}_{2} = ( {σ}_{*} = 1 , \overline{b} = [ 1 , 1 , … 0 ] ) \\ ⋮ & \\ {P}_{{2}^{L - 1}} = ℙ ( {C}_{{2}^{L - 1}} \vert {m}^{=} , {\overline{ϕ}}_{*} , σ ) , & {C}_{{2}^{L - 1}} = ( {σ}_{*} = 1 , \overline{b} = [ 1 , 1 , … , 1 ] ) \\ {P}_{{2}^{L - 1} + 1} = ℙ ( {C}_{{2}^{L - 1} + 1} \vert {m}^{=} , {\overline{ϕ}}_{*} , θ ) , & {C}_{{2}^{L - 1} + 1} = ( {σ}_{*} = 2 , \overline{b} = [ 0 , 1 , … , 0 ] ) \\ ⋮ & \\ {P}_{2 * {2}^{L - 1}} = ℙ ( {C}_{2 * {2}^{L - 1}} \vert {m}^{=} , {\overline{ϕ}}_{*} , θ ) , & {C}_{2 * {2}^{L - 1}} = ( {σ}_{*} = 2 , \overline{b} = [ 1 , 1 , … , 1 ] ) \\ ⋮ & \\ {P}_{L * {2}^{L - 1}} = ℙ ( {C}_{L * {2}^{L - 1}} \vert {m}^{=} , {\overline{ϕ}}_{*} , θ ) , & {C}_{L * {2}^{L - 1}} = ( {σ}_{*} = L , \overline{b} = [ 1 , 1 , … , 1 ] )\end{matrix}$$

constructing the Categorical distribution and sampling the initial condition gene state:

$${σ}_{*} , \overline{b} \vert {m}^{=} , \overline{q} , θ \sim {Categorical}_{[ {C}_{1} , {C}_{2} , … , {C}_{L * {2}^{L - 1}} ]} ( {P}_{1} , {P}_{2} , … , {P}_{L * {2}^{L - 1}} ) .$$

The conditional probabilities can be written as:

$$ℙ ( {σ}_{*} = {l}^{′} , \overline{b} \vert {m}^{=} , \overline{q} , θ ) ∝ ℙ ( {m}^{=} \vert {σ}_{*} , \overline{b} , θ ) ℙ ( \overline{b} \vert \overline{q} , {σ}_{*} ) ℙ ( {σ}_{*} \vert \overline{q} ) = {{\prod }^{K}}_{k = 1} {{\prod }^{{J}_{k}}}_{j = 1} ( {{\sum }^{L}}_{l = 1} {P}^{t , {l}^{′} , \overline{b} , θ} ( {σ}_{l} , {m}_{k}^{j} ) ) \times {{\prod }^{L}}_{l} {( {δ}_{{l}^{′} l} + ( 1 - {δ}_{{l}^{′} l} ) {q}_{l} )}^{{b}_{l}} {( 1 - {δ}_{{l}^{′} l} + ( 1 - {δ}_{{l}^{′} l} ) {q}_{l} )}^{1 - {b}_{l}} ( \frac{{q}_{{l}^{′}}}{{\sum }_{l = 1}^{L} {q}_{l}} ) .$$

The logarithm of the conditional probability can be written as:

$$log = ( ℙ ( {σ}_{*} = {l}^{′} , \overline{b} \vert {m}^{=} , \overline{q} , θ ) ) = {{\sum }^{K}}_{k - 1} {{\sum }^{{J}_{k}}}_{j = 1} log ( {{\sum }^{L}}_{l = 1} {P}^{t , {l}^{′} , θ} ( {σ}_{l} , {m}_{k}^{j} ) ) + {{\sum }^{L}}_{l = 1} ( {b}_{l} ( log ( {δ}_{{l}^{′} l} + ( 1 - {δ}_{{l}^{′} l} ) ) {q}_{l} ) ) + ( 1 - {b}_{l} ) ( log ( 1 - ( {δ}_{{l}^{′} l} + ( 1 - {δ}_{{l}^{′} l} ) {q}_{l} ) ) ) ) + log ( {q}_{{l}^{′}} ) - log ( {{\sum }^{L}}_{l = 1} {q}_{l} ) .$$

The system can employ a mixture of MH and Hamiltonian Monte Carlo (HMC) sampling schemes to sample the reaction rates. Both cases involve sampling from the marginal posterior distribution:

$$ℙ ( \tilde{θ} \vert {m}^{=} , \overline{q} , {σ}_{*} , \overline{b} ) ∝ ℙ ( {m}^{=} \vert {σ}_{*} , \overline{b} , \tilde{θ} ) ℙ ( \tilde{θ} ) .$$

### 5.6.2 Sampling Success Probabilities

The system can employ a Metropolis-Hastings (MH) sampling scheme to sample the initial success probability vector, q̅ from the marginal posterior distribution:

$$ℙ ( {q}_{l} , \vert {m}^{=} , {\overline{σ}}_{*} , \overline{b} , θ ) ∝ ℙ ( \overline{b} \vert {σ}_{*} , \overline{q} ) ℙ ( {σ}_{*} \vert \overline{q} ) ℙ ( \overline{q} ) = {{\prod }^{L}}_{l = 1} Bernoulli ( {b}_{l} ; {δ}_{{σ}_{*} l} + ( 1 - {δ}_{{σ}_{*} l} ) {q}_{l} ) Categorical ( {σ}_{*} ; \frac{\overline{q}}{{\sum }_{l = 1}^{L} {q}_{l}} ) \times {{\prod }^{L}}_{l = 1} Beta ( {q}_{l} ; \frac{ζ}{L} , \frac{L - 1}{L} ) = {{\prod }^{L}}_{l} {( {δ}_{{σ}_{*} l} + ( 1 - {δ}_{{σ}_{*} l} ) {q}_{l} )}^{{b}_{l}} {( 1 - ( {δ}_{{σ}_{*} l} + ( 1 - {δ}_{{σ}_{*} l} ) {q}_{l} ) )}^{1 - {b}_{l}} ( \frac{{q}_{{σ}_{*}}}{{\sum }_{l = 1}^{L} {q}_{l}} ) \times {{\prod }^{L}}_{l = 1} \frac{1}{B ( \frac{ζ}{L} , \frac{L - 1}{L} )} {{q}_{l}^{\frac{ζ}{L} - 1} ( 1 - {q}_{l} )}^{\frac{L - 1}{L}} .$$

The system can take proposals within the MH sampling scheme directly from the prior distribution, giving an acceptance ratio of:

$${R}_{{\overline{q}}^{old}} ( {\overline{q}}^{prop} ) = \frac{ℙ ( ( {\overline{b}}^{old} ) \vert {σ}_{*}^{old} , {\overline{q}}^{prop} ) ℙ ( {σ}_{*}^{old} \vert {\overline{q}}^{prop} )}{ℙ ( {\overline{b}}^{old} \vert {σ}_{*}^{old} , {\overline{q}}^{old} ) ℙ ( {σ}_{*}^{old} \vert {\overline{q}}^{old} )} = \frac{{{\prod }^{L}}_{l} {( {δ}_{{σ}_{*} l} + ( 1 - {δ}_{{σ}_{*} l} ) {q}_{l}^{prop} )}^{{b}_{l}^{old}} {( 1 - ( {δ}_{{σ}_{*} l} + ( 1 - {δ}_{{σ}_{*} l} ) {q}_{l}^{prop} ) )}^{1 - {b}_{l}^{old}}}{{{\prod }^{L}}_{l} {( {δ}_{{σ}_{*} l} + ( 1 - {δ}_{{σ}_{*} l} ) {q}_{l}^{old} )}^{{b}_{l}^{old}} {( 1 - ( {δ}_{{σ}_{*} l} + ( 1 - {δ}_{{σ}_{*} l} ) {q}_{l}^{old} ) )}^{1 - {b}_{l}^{old}}} \times \frac{\frac{{q}_{{σ}_{*}}^{prop}}{{{\sum }^{L}}_{l = 1} {q}_{l}^{prop}}}{\frac{{q}_{{σ}_{*}}^{old}}{{{\sum }^{L}}_{l = 1} {q}_{l}^{old}}} .$$

In log form:

$$log ( {R}_{{\overline{q}}^{old}} ( {\overline{q}}^{prop} ) ) = {{\sum }^{L}}_{l} ( {b}_{l}^{old} ( log ( {δ}_{{σ}_{*} l} + ( 1 - {δ}_{{σ}_{*} l} ) {q}_{l}^{prop} ) - log ( {δ}_{{σ}_{*} l} + ( 1 - {δ}_{{σ}_{*} l} ) {q}_{l}^{old} ) ) + ( 1 - {b}_{l}^{old} ) ( log ( 1 - {δ}_{{σ}_{*} l} + ( 1 - {δ}_{{σ}_{*} l} ) {q}_{l}^{prop} ) - log ( 1 - {δ}_{{σ}_{*} l} + ( 1 - {δ}_{{σ}_{*} l} ) {q}_{l}^{old} ) ) ) + log ( {q}_{{σ}_{*}}^{prop} ) - log ( {{\sum }^{L}}_{l = 1} {q}_{l}^{prop} ) - log ( {q}_{{σ}_{*}}^{old} ) + log ( {{\sum }^{L}}_{l = 1} {q}_{l}^{old} ) .$$

### 5.6.3 Sampling the Initial Condition Gene State

The system can employ direct sampling within a Gibbs sampling scheme to sample the initial condition, σ_{*} from the marginal posterior distribution:

$$ℙ ( {σ}_{*} \vert {m}^{=} , \overline{q} , \overline{b} , θ ) ∝ ℙ ( {m}^{=} \vert {σ}_{*} , \overline{b} , θ ) ℙ ( \overline{b} \vert \overline{q} , {σ}_{*} ) ℙ ( {σ}_{*} \vert \overline{q} ) = {{\prod }^{K}}_{k = 1} {{\prod }^{{J}_{k}}}_{j = 1} ( {{\sum }^{L}}_{l = 1} {P}^{t , {σ}_{*} , \overline{b} , θ} ( {σ}_{l} , {m}_{k}^{j} ) ) {{\prod }^{L}}_{l} Bernoulli ( {b}_{l} ; {δ}_{{σ}_{*} l} + ( 1 - {δ}_{{σ}_{*} l} ) {q}_{l} ) \times Categorical ( {σ}_{*} ; \frac{\overline{q}}{{\sum }_{l = 1}^{L} {q}_{l}} ) = {{\prod }^{K}}_{k = 1} {{\prod }^{{J}_{k}}}_{j = 1} ( {{\sum }^{L}}_{l = 1} {P}^{t , {σ}_{*} , \overline{b} , θ} ( {σ}_{l} , {m}_{k}^{j} ) ) \times {{\prod }^{L}}_{l} {( {δ}_{{σ}_{*} l} + ( 1 - {δ}_{{σ}_{*} l} ) {q}_{l} )}^{{b}_{l}} {( 1 - {δ}_{{σ}_{*} l} + ( 1 - {δ}_{{σ}_{*} l} ) {q}_{l} )}^{1 - {b}_{l}} ( \frac{{q}_{{σ}_{*}}}{{\sum }_{l = 1}^{L} {q}_{l}} ) .$$

The system can sample the initial condition gene state through posteriors of all possible gene states:

$$\begin{matrix}{σ}_{*} = 1 , {P}_{1} = ℙ ( {σ}_{*} = 1 \vert {m}^{=} , {\overline{ϕ}}_{*} , θ ) \\ {σ}_{*} = 2 , {P}_{2} = ℙ ( {σ}_{*} = 2 \vert {m}^{=} , {\overline{ϕ}}_{*} , θ ) \\ ⋮ \\ {σ}_{*} = L , {P}_{L} = ℙ ( {σ}_{*} = L \vert {m}^{=} , {\overline{ϕ}}_{*} , θ )\end{matrix}$$

constructing the Categorical distribution and sampling the initial condition gene state:

$${σ}_{*} \vert {m}^{=} , \overline{q} , \overline{b} , θ \sim {Categorical}_{[ {σ}_{1} , {σ}_{2} , … {σ}_{L} ]} ( {P}_{1} , {P}_{2} , … , {P}_{L} ) .$$

The conditional probabilities can be written as:

$$ℙ ( {σ}_{*} = {l}^{′} \vert {m}^{=} , \overline{q} , \overline{b} , θ ) ∝ ℙ ( {m}^{=} \vert {σ}_{*} , \overline{b} , θ ) ℙ ( \overline{b} \vert \overline{q} , {σ}_{*} ) ℙ ( {σ}_{*} \vert \overline{q} ) = {{\prod }^{K}}_{k = 1} {{\prod }^{{J}_{k}}}_{j = 1} ( {{\sum }^{L}}_{l = 1} {P}^{t , {l}^{′} , \overline{b} , θ} ( {σ}_{l} , {m}_{k}^{j} ) ) \times {{\prod }^{L}}_{l} {( {δ}_{{l}^{′} l} + ( 1 - {δ}_{{l}^{′} l} ) {q}_{l} )}^{{b}_{l}} {( {δ}_{{l}^{′} l} + ( 1 - {δ}_{{l}^{′} l} ) {q}_{l} )}^{1 - {b}_{l}} ( \frac{{q}_{{l}^{′}}}{{\sum }_{l = 1}^{L} {q}_{l}} ) .$$

The logarithm of the conditional probability is:

$$log = ( ℙ ( {σ}_{*} = {l}^{′} , \overline{b} \vert {m}^{=} , \overline{q} , \overline{b} , θ ) ) = {{\sum }^{K}}_{k - 1} {{\sum }^{{J}_{k}}}_{j = 1} log ( {{\sum }^{L}}_{l = 1} {P}^{t , {l}^{′} , θ} ( {σ}_{l} , {m}_{k}^{j} ) ) + {{\sum }^{L}}_{l = 1} ( {b}_{l} ( log ( {δ}_{{l}^{′} l} + ( 1 - {δ}_{{l}^{′} l} ) ) {q}_{l} ) ) + ( 1 - {b}_{l} ) log ( 1 - ( {δ}_{{l}^{′} l} + ( 1 - {δ}_{{l}^{′} l} ) {q}_{l} ) ) ) + log ( {q}_{{l}^{′}} ) - log ( {{\sum }^{L}}_{l = 1} {q}_{l} ) .$$

### 5.6.4 Sampling of the Loads

The system can apply direct sampling within a Gibbs sampling scheme to sample the loads b from the marginal posterior distribution:

$$ℙ ( \overline{b} \vert {m}^{=} , \overline{q} , {σ}_{*} , \overline{k} , \overline{β} , γ ) ∝ ℙ ( {m}^{=} \vert {σ}_{*} , \overline{b} , θ ) ℙ ( \overline{b} \vert \overline{q} , {σ}_{*} ) = {{\prod }^{K}}_{k = 1} {{\prod }^{{J}_{k}}}_{j = 1} ( {{\sum }^{L}}_{l = 1} {P}^{t , {σ}_{*} , \overline{b} , θ} ( {σ}_{l} , {m}_{k}^{j} ) ) {{\prod }^{L}}_{l} Bernoulli ( {b}_{l} ; {δ}_{{σ}_{*} l} + ( 1 - {δ}_{{σ}_{*} l} ) {q}_{l} ) = {{\prod }^{K}}_{k = 1} {{\prod }^{{J}_{k}}}_{j = 1} ( {{\sum }^{L}}_{l = 1} {P}^{t , {σ}_{*} , \overline{b} , θ} ( {σ}_{l} , {m}_{k}^{j} ) ) \times {{\prod }^{L}}_{l} {( {δ}_{{σ}_{*} l} + ( 1 - {δ}_{{σ}_{*} l} ) {q}_{l} )}^{{b}_{l}} {( 1 - {δ}_{{σ}_{*} l} + ( 1 - {δ}_{{σ}_{*} l} ) {q}_{l} )}^{1 - {b}_{l}} .$$

To sample the loads using direct sampling within the Gibbs sampling scheme, the system samples the loads simultaneously through posteriors of all configurations of loads:

$$\begin{matrix}{P}_{1} = ℙ ( {B}_{1} \vert {m}^{=} , {\overline{ϕ}}_{*} , {σ}_{*} , \overline{k} , \overline{β} , γ ) , & {B}_{1} = [ 0 , 0 , … , 0 ] \\ {P}_{2} = ℙ ( {B}_{2} \vert {m}^{=} , {\overline{ϕ}}_{*} , {σ}_{*} , \overline{k} , \overline{β} , γ ) , & {B}_{2} = [ 1 , 0 , … , 0 ] \\ ⋮ & \\ {P}_{{2}^{L}} = ℙ ( {B}_{{2}^{L}} \vert {m}^{=} , {\overline{ϕ}}_{*} , {σ}_{*} , \overline{k} , \overline{β} , γ ) & {B}_{{2}^{L}} = [ 1 , 1 , … , 1 ]\end{matrix}$$

constructing the Categorical distribution and sampling the configuration of loads:

$$\overline{b} \vert {m}^{=} , \overline{q} , {σ}_{*} , θ \sim {Categorical}_{[ {B}_{1} , {B}_{2} , … , {B}_{{2}^{L}} ]} ( {P}_{1} , {P}_{2} , … , {P}_{{2}^{L}} ) .$$

However, since the Categorical distribution has 2^{L} arguments, the computational cost can be lessened by sampling a smaller set of loads. A random set of loads b̅ₗ, are defined such that l′≠σ_{*} for all l′. The system can apply direct sampling to these. The posterior over this smaller set of nodes to be updated simultaneously is then: [symbol] (b̅_{l′}|m̅, q̅, σ_{*,} b̅_{-l′}, k̅, β̅, γ) in which b̅_{-l′} is the set b̅ excluding the loads contained in b̅_{l′}. Thus, the conditional probability can then be written as:

$$\begin{matrix}ℙ ( {b}_{{l}^{′}} \vert {m}^{=} , \overline{q} , {σ}_{*} , {\overline{b}}_{- {l}^{′}} , θ ) ∝ ℙ ( {m}^{=} \vert {σ}_{*} , \overline{b} , θ ) ℙ ( \overline{b} \vert \overline{q} , {l}^{′} ) \\ = {\prod }_{k = 1}^{K} {\prod }_{j = 1}^{{J}_{k}} ( {\sum }_{l = 1}^{L} {P}^{t , {σ}_{*} , \overline{b} , θ} ( {σ}_{l} , {m}_{k}^{j} ) ) {\prod }_{l}^{L} Bernoulli ( {b}_{l} ; {δ}_{{σ}_{*} l} + ( 1 - {δ}_{{σ}_{*} l} ) {q}_{l} ) \\ = {\prod }_{k = 1}^{K} {\prod }_{j = 1}^{{J}_{k}} ( {\sum }_{l = 1}^{L} {P}^{t , {σ}_{*} , \overline{b} , θ} ( {σ}_{l} , {m}_{k}^{j} ) ) \\ \times {\prod }_{l}^{L} {( {δ}_{{l}^{′} l} + ( 1 - {δ}_{{σ}_{*} l} ) {q}_{l} )}^{{b}_{l}} {( 1 - {δ}_{{σ}_{*} l} + ( 1 - {δ}_{{σ}_{*} l} ) {q}_{l} )}^{1 - {b}_{l}} ,\end{matrix}$$

The logarithm of the conditional probability is:

$$log = ( ℙ ( {\overline{b}}_{l} , \vert {m}^{=} , \overline{q} , {σ}_{*} , {\overline{b}}_{- {l}^{′}} , θ ) ) = {\sum }_{k - 1}^{K} {\sum }_{j = 1}^{J κ} log ( {\sum }_{l = 1}^{L} {P}^{t , {σ}_{*} , {b}_{- {l}^{′}} , θ} ( {σ}_{l} , {m}_{k}^{j} ) ) + {\sum }_{{l}^{′}} ( {b}_{{l}^{′}} ( log ( {δ}_{{σ}_{*} {l}^{′}} + ( 1 - {δ}_{{σ}_{*} {l}^{′}} ) ) {q}_{{l}^{′}} ) ) + ( 1 - {b}_{{l}^{′}} ) ( log ( 1 - ( {δ}_{{σ}_{*} {l}^{′}} + ( 1 - {δ}_{{σ}_{*} {l}^{′}} ) {q}_{{l}^{′}} ) ) ) ) .$$

### 5.6.5 Metropolis Hastings Sampling Scheme

The system can propose new samples using a multivariate normal distribution demonstrated below for the parameter \tilde{β}₁ and \tilde{γ},

$${ℚ}_{{\tilde{β}}_{l}^{old} {\tilde{γ}}^{old}} ( {\tilde{β}}_{l}^{prop} , {\tilde{γ}}^{prop} ) = MVNormal ( ( \begin{matrix}{\tilde{β}}_{l}^{prop} \\ {\tilde{γ}}^{prop}\end{matrix} ) ; ( \begin{matrix}{\tilde{β}}_{l}^{old} \\ {\tilde{γ}}^{old}\end{matrix} ) , {\sum }_{[ {2}_{l} , 3 ]} )$$

where Σ[₂_{ₗ}_{, 3}] is an adapted covariance matrix of size 2×2 with the corresponding pieces of the larger N×N covariance matrix Σ for βₗ, and γ. Continuing with this example, the acceptance ratio would then be:

$${R}_{{\tilde{β}}_{l}^{old} {\tilde{γ}}^{old}} ( {\tilde{β}}_{l}^{prop} , {\tilde{γ}}^{prop} ) = \frac{ℙ ( {m}^{=} \vert {l}_{*}^{old} , {\overline{b}}^{old} , {\tilde{θ}}^{prop} ) ℙ ( {\tilde{θ}}^{prop} )}{ℙ ( {m}^{=} \vert {σ}_{*}^{old} , {\overline{b}}^{old} , {\tilde{θ}}^{old} ) ℙ ( {\tilde{θ}}^{old} )} = \frac{{Π}_{k = 1}^{K} {Π}_{j = 1}^{J κ} ( {\sum }_{i = 1}^{L} {P}^{t , {σ}_{*}^{old} , {\overline{b}}^{old} , {10}^{{\tilde{θ}}^{prop}}} ( {σ}_{i} , {m}_{k}^{j} ) )}{{Π}_{k = 1}^{K} {Π}_{j = 1}^{J κ} ( {\sum }_{i = 1}^{L} {P}^{t , {σ}_{*}^{old} , {\overline{b}}^{old} , {10}^{{\tilde{θ}}^{old}}} ( {σ}_{i} , {m}_{k}^{j} ) )} \times \frac{Normal ( {\tilde{β}}_{l ′}^{prop} , {ϕ}_{{2}_{l}} , {ψ}_{{2}_{l}} ) Normal ( {\tilde{γ}}^{p r o p} ; {ϕ}_{3} , {ψ}_{3} )}{Normal ( {\tilde{β}}_{l}^{old} , {ϕ}_{{2}_{l}} , {ψ}_{{2}_{l}} ) Normal ( {\tilde{γ}}^{∘ l d} ; {ϕ}_{3} , {ψ}_{3} )} \times \frac{{ℚ}_{{\tilde{β}}_{l}^{prop} , {\tilde{γ}}^{prop}} ( {\tilde{β}}_{l}^{old} , {\tilde{γ}}^{old} )}{{ℚ}_{{\tilde{β}}_{l}^{old} , {\tilde{γ}}^{old}} ( {\tilde{β}}_{l}^{prop} , {\tilde{γ}}^{prop} )} = \frac{{\prod }_{k = 1}^{K} {\prod }_{j = 1}^{J κ} ( {\sum }_{i = 1}^{L} {P}^{t , {σ}_{*}^{old} , {\overline{b}}^{old} , {10}^{{\tilde{θ}}^{prop}}} ( {σ}_{i} , {m}_{k}^{j} ) )}{{\prod }_{k = 1}^{K} {\prod }_{j = 1}^{J κ} ( {\sum }_{i = 1}^{L} {P}^{t , {σ}_{*}^{old} , {\overline{b}}^{old} , {10}^{{\tilde{θ}}^{old}}} ( {σ}_{i} , {m}_{k}^{j} ) )} \times \frac{{e}^{- \frac{1}{2} {( \frac{{\tilde{β}}_{l}^{prop} - {ϕ}_{{2}_{l}}}{{ψ}_{{2}_{l}}} )}^{2}} {e}^{- \frac{1}{2} {( \frac{{\tilde{γ}}^{prop} - {ϕ}_{3}}{{ψ}_{3}} )}^{2}}}{{e}^{- \frac{1}{2} {( \frac{{\tilde{β}}_{l}^{old} - {ϕ}_{{2}_{l}}}{{ψ}_{{2}_{l}}} )}^{2}} {e}^{- \frac{1}{2} {( \frac{{\tilde{γ}}^{o l d} - {ϕ}_{3}}{{ψ}_{3}} )}^{2}}} \times \frac{{e}^{- \frac{1}{2} {( [ \begin{matrix}{\tilde{β}}_{l}^{old} \\ {\tilde{γ}}^{old}\end{matrix} ] - [ \begin{matrix}{\tilde{β}}_{l}^{prop} \\ {\tilde{γ}}^{prop}\end{matrix} ] )}^{T} {\sum }_{[ {2}_{l} , 3 ]}^{- 1} ( [ \begin{matrix}{\tilde{β}}_{l}^{old} \\ {\tilde{γ}}^{old}\end{matrix} ] - [ \begin{matrix}{\tilde{β}}_{l}^{prop} \\ {\tilde{γ}}^{prop}\end{matrix} ] )}}{{e}^{- \frac{1}{2} {( [ \begin{matrix}{\tilde{β}}_{l}^{prop} \\ {\tilde{γ}}^{prop}\end{matrix} ] - [ \begin{matrix}{\tilde{β}}_{l}^{old} \\ {\tilde{γ}}^{old}\end{matrix} ] )}^{T} {\sum }_{[ {2}_{l} , 3 ]}^{- 1} ( [ \begin{matrix}{\tilde{β}}_{l}^{old} \\ {\tilde{γ}}^{old}\end{matrix} ] - [ \begin{matrix}{\tilde{β}}_{l}^{prop} \\ {\tilde{γ}}^{prop}\end{matrix} ] )}} .$$

In log form this becomes:

$$\begin{matrix}log ( {R}_{{\tilde{β}}_{l}^{old} , {\tilde{γ}}^{old}} ( {\tilde{β}}_{l}^{prop} , {\tilde{γ}}^{prop} ) ) = {\sum }_{k - 1}^{K} {\sum }_{j = 1}^{J κ} log ( {\sum }_{i = 1}^{L} {P}^{t , {σ}_{*}^{old} , {\overline{b}}^{old} , {10}^{{\tilde{θ}}^{prop}}} ( {σ}_{i} , {m}_{k}^{j} ) ) , \\ {\sum }_{k - 1}^{K} {\sum }_{j = 1}^{J κ} log ( {\sum }_{i = 1}^{L} {P}^{t , {σ}_{*}^{old} , {\overline{b}}^{old} , {10}^{{\tilde{θ}}^{old}}} ( {σ}_{i} , {m}_{k}^{j} ) ) , \\ - \frac{1}{2} {( \frac{{\tilde{β}}_{l}^{prop} - {ϕ}_{{2}_{l}}}{{ψ}_{{2}_{l}}} )}^{2} - \frac{1}{2} {( \frac{{\tilde{γ}}^{prop} - {ϕ}_{3}}{{ψ}_{3}} )}^{2} \\ + \frac{1}{2} {( \frac{{\tilde{β}}_{l}^{prop} - {ϕ}_{{2}_{l}}}{{ψ}_{{2}_{l}}} )}^{2} - \frac{1}{2} {( \frac{{\tilde{γ}}^{prop} - {ϕ}_{3}}{{ψ}_{3}} )}^{2} \\ - \frac{1}{2} {( [ \begin{matrix}{\tilde{β}}_{l}^{old} \\ {\tilde{γ}}^{old}\end{matrix} ] - [ \begin{matrix}{\tilde{β}}_{l}^{prop} \\ {\tilde{γ}}^{prop}\end{matrix} ] )}^{T} {\sum }_{[ {2}_{l} , 3 ]}^{- 1} ( [ \begin{matrix}{\tilde{β}}_{l}^{old} \\ {\tilde{γ}}^{old}\end{matrix} ] - [ \begin{matrix}{\tilde{β}}_{l}^{prop} \\ {\tilde{γ}}^{prop}\end{matrix} ] ) \\ + \frac{1}{2} {( [ \begin{matrix}{\tilde{β}}_{l}^{prop} \\ {\tilde{γ}}^{prop}\end{matrix} ] - [ \begin{matrix}{\tilde{β}}_{l}^{old} \\ {\tilde{γ}}^{old}\end{matrix} ] )}^{T} {\sum }_{[ {2}_{l} , 3 ]}^{- 1} ( [ \begin{matrix}{\tilde{β}}_{l}^{old} \\ {\tilde{γ}}^{old}\end{matrix} ] - [ \begin{matrix}{\tilde{β}}_{l}^{prop} \\ {\tilde{γ}}^{prop}\end{matrix} ] )\end{matrix}$$

### 5.6.6 Hamiltonian Monte Carlo Sampling Scheme

The sampling method used here is the same as in the linear space, however the present disclosure defines the Hamiltonian systems based upon the new system. Therefore, \tilde{q}=\tilde{θ}, and the Hamiltonian function is:

$$\begin{matrix}H ( \tilde{q} , p ) = L ( \tilde{q} ) + V ( \tilde{q} ) + T ( p ) , \\ L ( \tilde{q} ) = - ( log ( ℙ ( \tilde{q} ) ) ) \\ = {\sum }_{n = 1}^{N} log ( Normal ( {\tilde{q}}_{n} ; {ϕ}_{n} ; {ψ}_{n} ) ) , \\ = log ( ψ \sqrt{2 π} ) + \frac{1}{2} {( \frac{{\tilde{q}}_{n} - {ϕ}_{n}}{{ψ}_{n}} )}^{2} \\ V ( \tilde{q} ) = {\sum }_{k = 1}^{K} {\sum }_{j = 1}^{J κ} log ( {\sum }_{i = 1}^{L} {P}^{t , {σ}_{*}^{old} , {\overline{b}}^{old} , {10}^{∼ prop}} ( {σ}_{i} , {m}_{k}^{j} ) ) ,\end{matrix}$$

and T(P) is the same as in the linear space.

In order to advance a half step forward using H¹(\tilde{q}, p), the present disclosure now considers the change of variables \tilde{q}=log₁₀(q) to find:

$$\frac{\partial V ( \tilde{q} )}{\partial \tilde{q}} = \frac{\partial V ( q )}{\partial q} \frac{\partial q}{\partial \tilde{q}} = 1 {0}^{\tilde{q}} log ( 10 ) \frac{\partial V ( q )}{\partial q} , where \frac{\partial V ( q )}{\partial q}$$

is the same as V_{q}(q) above.

In order to advance the full step forward using H²(\tilde{q}), use L_{\tilde{q}}(\tilde{q}), demonstrated for the rates \tilde{β}ₗ and \tilde{γ},

$${L}_{\tilde{q}} ( \tilde{q} ) = ( \begin{matrix}\frac{{\tilde{β}}_{l} - {ϕ}_{{2}_{l}}}{{ψ}_{{2}_{l}}^{2}} \\ \frac{\tilde{γ} - {ϕ}_{3}}{{ψ}_{3}^{2}}\end{matrix} ) .$$

To approximate, a second order and symplectic implicit midpoint rule integration can be applied using custom M_{ATLAB} code:

$$\frac{{\tilde{q}}^{new} - {\tilde{q}}^{old}}{h} = {M}^{- 1} ( \frac{{p}^{old} + {p}^{new}}{2} ) \frac{{p}^{new} - {p}^{old}}{h} = {L}_{\tilde{q}} ( \frac{{\tilde{q}}^{old} + {\tilde{q}}^{new}}{2} ) \qquad ( 1 )$$

Solving for P^{new}, and demonstrating with the parameter \tilde{β}ₗ:

$${p}_{{2}_{l}}^{new} = h ( \frac{( \frac{{\tilde{β}}_{l}^{old} + {\tilde{β}}_{l}^{new}}{2} ) - {ϕ}_{{2}_{l}}}{{ψ}_{{2}_{l}}^{2}} ) + {p}_{{2}_{l}}^{old} . \qquad ( 2 )$$

Plugging Eq. (2) into Eq. (1) and following these algebraic steps,

$$\frac{{\tilde{β}}_{l}^{new} - {\tilde{β}}_{l}^{old}}{h} = \frac{1}{{m}_{{2}_{l}}} ( \frac{{p}_{{2}_{l}}^{old} + ( h \frac{( \frac{{\tilde{β}}_{l}^{old} - {\tilde{β}}_{l}^{new}}{2} ) - {ϕ}_{{2}_{l}}}{{ψ}_{{2}_{l}}^{2}} + {p}_{{2}_{l}}^{old} )}{2} )$$

$$\frac{{\tilde{β}}_{l}^{new} - {\tilde{β}}_{l}^{old}}{h} = \frac{1}{{m}_{{2}_{l}}} ( {p}_{{2}_{l}}^{old} + \frac{h}{2 {ψ}^{2}} ( \frac{{\tilde{β}}_{l}^{old} - {\tilde{β}}_{l}^{new}}{2} - {ϕ}_{{2}_{l}} ) )$$

$${\tilde{β}}_{l}^{new} - {\tilde{β}}_{l}^{old} = \frac{h}{{m}_{{2}_{l}}} {p}_{{2}_{l}}^{old} + \frac{{h}^{2}}{2 {ψ}^{2} {m}_{{2}_{l}}} ( \frac{{\tilde{β}}_{l}^{old} - {\tilde{β}}_{l}^{new}}{2} - {ϕ}_{{2}_{l}} )$$

$${\tilde{β}}_{l}^{new} - {\tilde{β}}_{l}^{old} = \frac{h}{{m}_{{2}_{l}}} {p}_{{2}_{l}}^{old} + \frac{{h}^{2}}{4 {ψ}^{2} {m}_{{2}_{l}}} {\tilde{β}}_{{2}_{l}}^{old} + \frac{{h}^{2}}{4 {ψ}^{2} {m}_{{2}_{l}}} {\tilde{β}}_{{2}_{l}}^{new} - \frac{{h}^{2}}{2 {ψ}^{2} {m}_{{2}_{l}}} {ϕ}_{{2}_{l}}$$

$${\tilde{β}}_{l}^{n e w} - \frac{{h}^{2}}{4 {ψ}^{2} {m}_{{2}_{l}}} {\tilde{β}}_{{2}_{l}}^{new} = \frac{h}{{m}_{{2}_{l}}} {p}_{{2}_{l}}^{old} + \frac{{h}^{2}}{4 {ψ}^{2} {m}_{{2}_{l}}} {\tilde{β}}_{{2}_{l}}^{old} - \frac{{h}^{2}}{2 {ψ}^{2} {m}_{{2}_{l}}} {ϕ}_{{2}_{l}} + {\tilde{β}}_{{2}_{l}}^{old} ,$$

the following can be arrived at:

$$\begin{matrix}{\tilde{β}}_{l}^{new} = \frac{\frac{h}{{m}_{{2}_{l}}} {p}_{{2}_{l}}^{old} - \frac{{h}^{2}}{2 {ψ}_{{2}_{l}}^{2} {m}_{{2}_{l}}} {ϕ}_{{2}_{l}} + ( 1 + \frac{{h}^{2}}{4 {ψ}_{{2}_{l}}^{2} {m}_{{2}_{l}}} ) {\tilde{β}}_{l}^{old}}{( 1 + \frac{{h}^{2}}{4 {ψ}_{{2}_{l}}^{2} {m}_{{2}_{l}}} )} \\ = \frac{\frac{4 {ψ}_{{2}_{l}}^{2} h}{{m}_{{2}_{l}}} {p}_{{2}_{l}}^{old} - \frac{2 {h}^{2}}{{m}_{{2}_{l}}} {ϕ}_{{2}_{l}} + ( 4 {ψ}_{{2}_{l}}^{2} + \frac{{h}^{2}}{{m}_{{2}_{l}}} ) {\tilde{β}}_{l}^{old}}{( 4 {ψ}_{{2}_{l}}^{2} - \frac{{h}^{2}}{{m}_{{2}_{l}}} )} .\end{matrix}$$

### 5.7 Robustness Analysis and Inference Validation on Synthetic Data

### 5.7.1 Maximum Transcription Rate

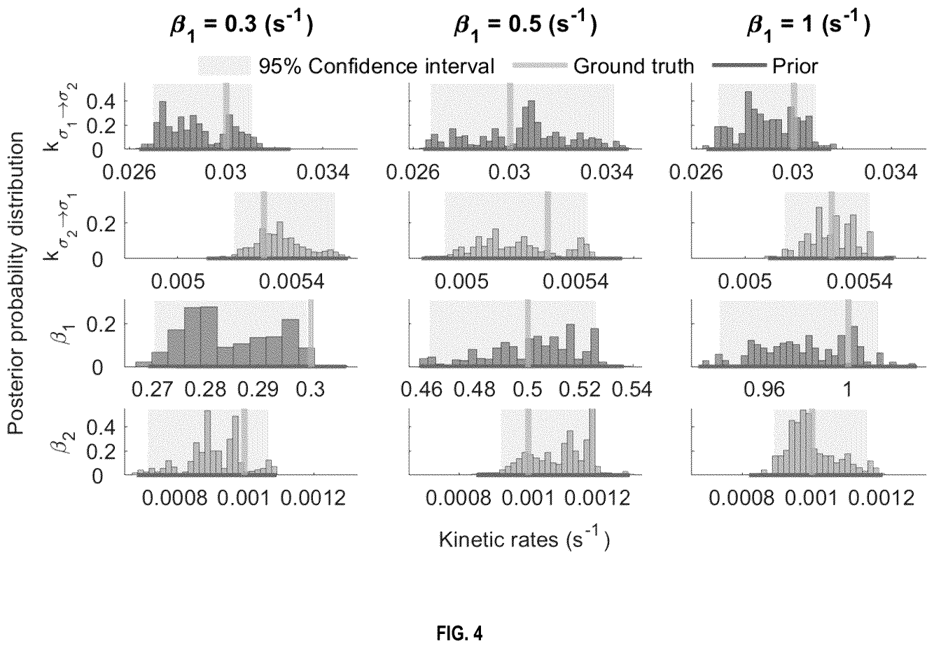

Due to the high number of model parameters, the present disclosure analyzes the method's sensitivity to changes in rates by varying the maximum production rate (β₁). If all other rates are maintained, changing β₁ corresponds to changing the expected number of RNA transcripts present in the cells. By varying β₁ from β₁=0.3 s⁻¹ to β₁=1 s⁻¹ (see FIG. 4), the present disclosure shows that the method's prediction accuracy is robust under changes in the true gene network's associated parameters.

### 5.7.2 Specification of RNA Degradation Rate

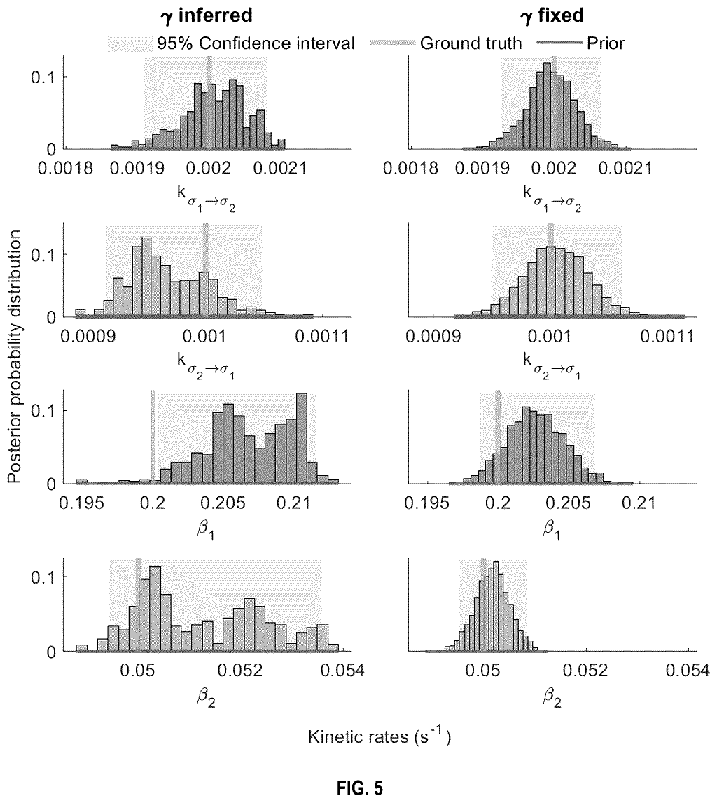

In general, it is difficult to predict whether the amount of data provided to the method is sufficient to infer all rates simultaneously. To wit, FIG. 5 shows how specifying one rate by hand allows smaller uncertainty in posteriors over the other rates.

### 6. Results

The examples above assume the availability of snapshot smFISH data containing RNA counts per cell, mₜ_{ₖ}^{j}, collected at time points, t_{1:K}, and cells indexed as j=1 , . . . , Jₖ. For simplicity, the RNA counts from all cells at all time points are denoted herein as m̅={{mₜ_{ₖ}^{j}}j=1:Jₖ}k=1: K. Using this information, a goal of the present disclosure is to predict the transcriptional gene output, i.e., through inference of both the gene states (that is, perform model selection) as well as inference of the associated rate parameters and thus gene state connectivity (parameter inference).

Here, the present disclosure shows ability of the methods to perform model selection and parameter inference for gene networks using both experimental and synthetic data. In BNPs, the model structure (which, in this case, corresponds to a number of gene states) is treated as a parameter, and can thus be inferred alongside all other parameters. The remaining parameters of interest are: the production rates, βₗ, for each gene state; the transition rates between various gene states, k_{σ}_{ₗ}_{→σ′}_{ₗ}, for l≠l′; and the RNA copy rate of degradation, γ. Since the system works within the BNP paradigm, parameter estimates are drawn from fully joint posterior probability distributions over rates as well as gene states, learned simultaneously and self-consistently. Samples from these posteriors are displayed below in the form of histograms. Naturally, these histograms provide a complete assessment of each quantity's value alongside their respective uncertainties.

First, the present disclosure demonstrates robustness of the method on synthetic data which replicates dynamics similar to experimental data sets, but covers a broader range of scenarios than are available experimentally. Subsequently, results are shown for experiments on the lacZ pathway in E. coli cells and the STL1 pathway in S. cerevisiae yeast cells.

### 6.1 Robustness Analysis

### 6.1.1 Number of States

In line with the current literature on gene expression, the method is evaluated on three different models consisting of one, two, and three gene states. In the two and three state models, one state has a production rate of zero.

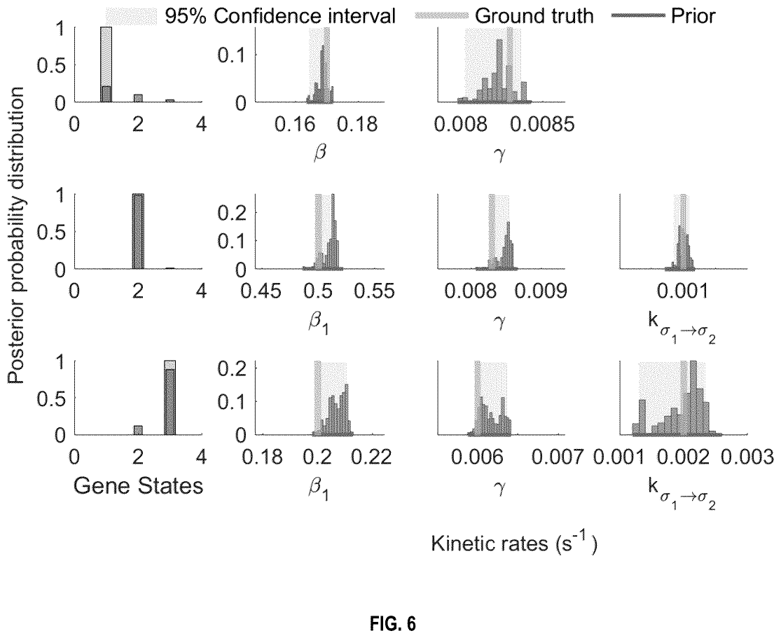

FIG. 6 shows the results of the method for one, two and three gene state models. As the methods provided herein work with synthetic data for which a ground truth is known, one can ascertain that the method is successful in placing substantial posterior probability on parameters close to ground truth.

Perhaps counter-intuitively for the simplest of gene networks, which should be easiest to infer, the posterior probability distributions over gene states are broader. The reason is subtle: models that estimate a greater number of gene states can approximate a one state model (but not vice versa) by having nearly identical production rates for each of the gene states. However, since the production rates of all possible gene states are not perfectly identical, the methods disclosed herein still favor the true number of states.

### 6.1.2 Quantity of Data

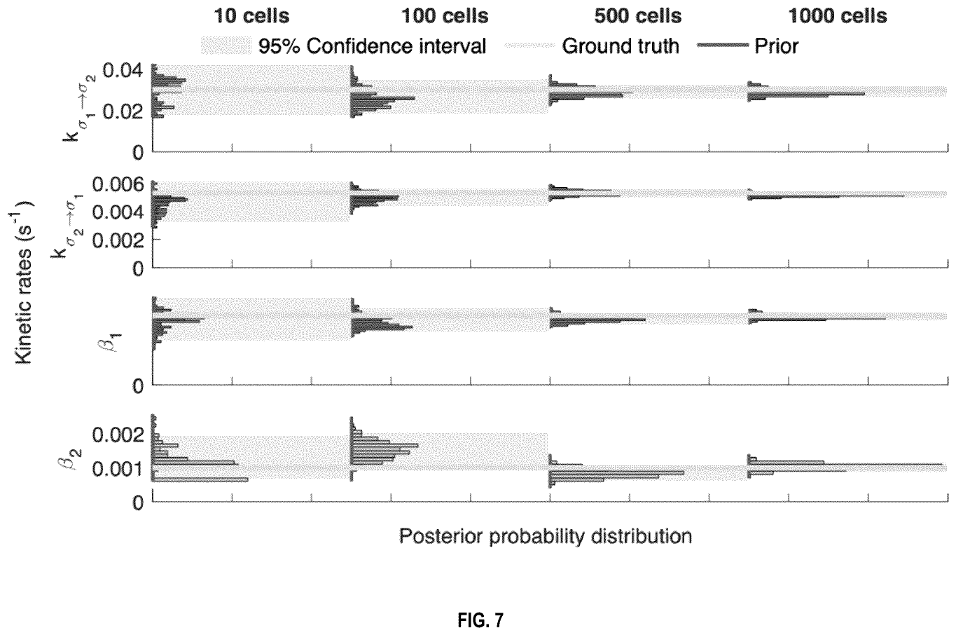

FIG. 7 demonstrates robustness of the method with respect to data set size. For networks with two gene states, the method makes accurate inference across three orders of magnitude in the number of cells extracted per time point. While it is shown that the precision of estimates (breadth of posteriors) made by the model does scale with the quantity of data, the accuracy does not. In fact, the method makes accurate inference on the two gene state model provided data on only a handful of cells, with posterior probabilities whose breadth reflect the small quantity of data.

For a detailed overview of the remaining robustness analysis of the method, refer to Section 5.7.

### 6.1.3 Synthetic Data Generation

Synthetic data used in Section 6.1 was generated using computer simulation based on Gillespie's Stochastic Simulation Algorithm. Details of the model are outlined in Section 5.1, with inference of all parameters detailed in Section 5.1.

### 6.2 Experimental Results

smFISH RNA count data from E. coli and S. cerevisiae cells are analyzed. For simplicity here, the RNA molecules' degradation rate is calibrated using previously-established results. FIG. 5 demonstrates that calibrating one rate, as expected, has the net effect of reducing uncertainty in the other rates inferred.

6.2.1 E. coli

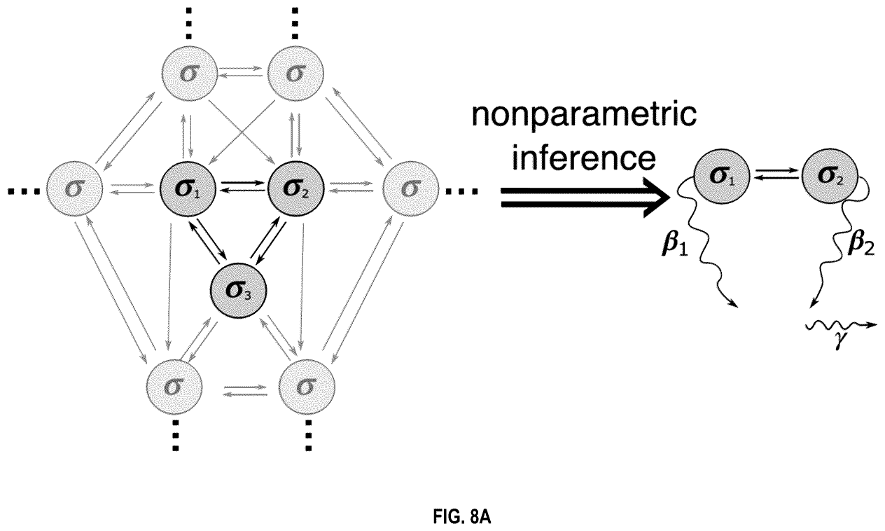

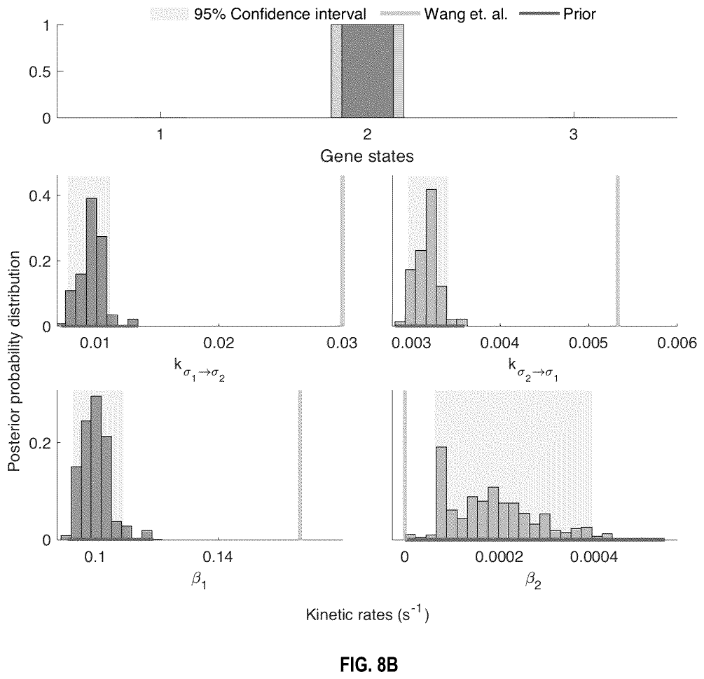

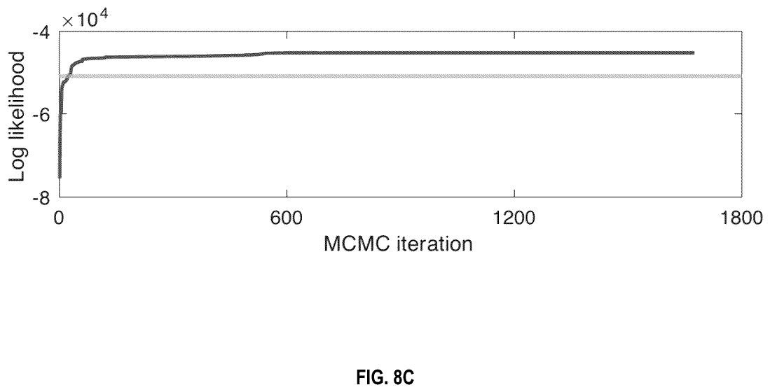

This section demonstrates simultaneous gene state number and parameter inference for the lacZ pathway in E. coli cells, grown in slow-growth media. Results are shown in FIGS. 8A-8C, which also shows point estimates obtained by another work from maximum likelihood. To be clear, the other work posits a model (i.e., pre-specify gene states) and, given the model, learn parameters from the data.

The states posited in the other work agree with what is learned directly from the data. In terms of parameters, general agreement is also found in the lowest production rate. However, disagreement of 70%, 40%, and 43% is found in parameters k_{σ}_{₁}_{→σ}_{₂}, k_{σ}_{₂}_{→σ}_{₁} and β₁ respectively, when comparing maximum a posteriori (MAP) estimate with respect to those reported in other works. This disagreement highlights a core issue: even when learning only rates (and positing states by hand) other methods cannot efficiently sample their high dimensional posterior. To wit, the methods can employ Hamiltonian Monte Carlo (HMC) and Parallel Tempering (PT) sampling schemes, allowing the method to avoid becoming trapped in local maxima. As a result, comparison of likelihoods dramatically favors the parameters to which the methods outlined herein have converged (by contrast to those reported in another work). Indeed, the MAP estimate (θ′) of the present method is more probable than that of other works (θ) by a factor of

$$ln ( \frac{P ( {m}^{=} \vert {θ}^{′} )}{P ( {m}^{=} \vert θ )} ) \approx 83 .$$

A trace of log likelihood of the present method surpassing that of another work can be seen in FIG. 8C.

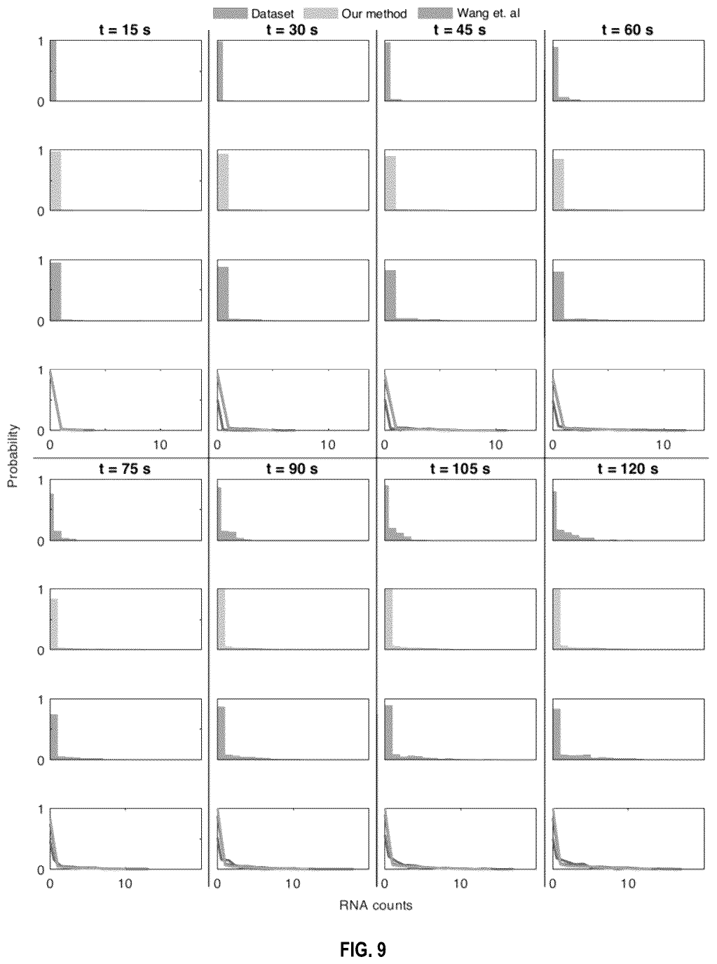

Interestingly, despite the significant difference between e and 0′, RNA count histograms across time points appear qualitatively similar; see FIG. 9. This is expected as static histograms do not contain temporal information leveraged by the present methods in analyzing time-ordered snapshot data. By the same token, it highlights the limitations of assessing kinetic rates and gene states by comparing RNA histograms across time points to the method proposed herein.

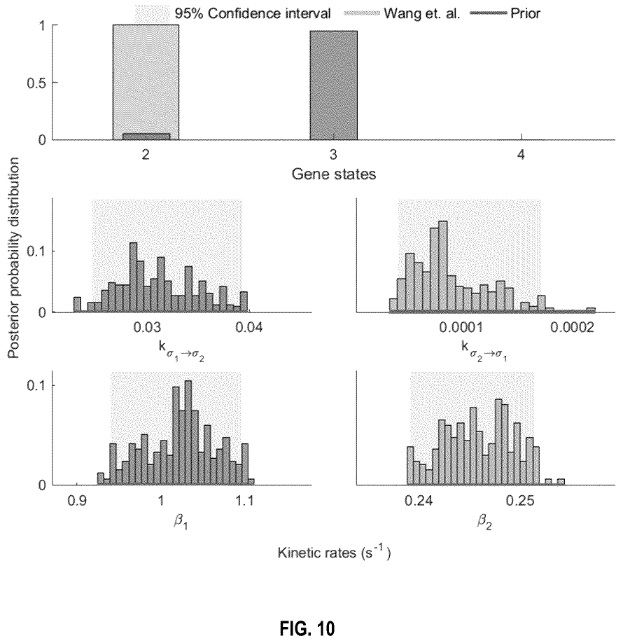

FIG. 10 compares a learned model obtained through the methods outlined herein to that estimated using other methods for E. coli grown in a fast-growth medium (glucose at 37° C.). The (full nonparametric) method outlined herein confidently infers three gene states, by contrast to other methods which assume two states. As different models are predicted, a direct likelihood comparison is more difficult here than in the previous case.

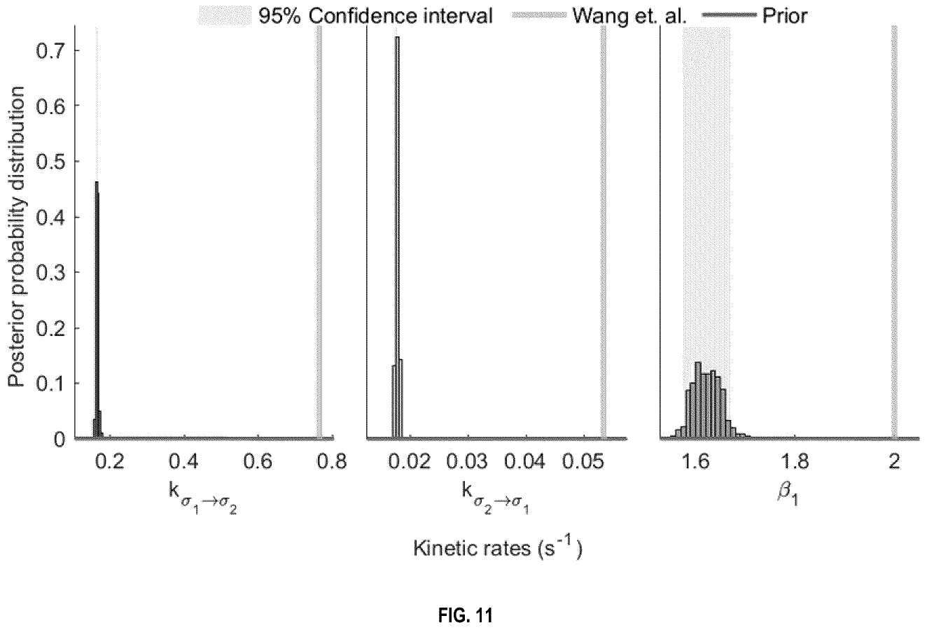

However, in order to directly compare likelihoods, the present method can be restricted to infer parameters by imposing by hand a two state gene expression model, with one production rate fixed at zero (FIG. 11). Disagreement of similar order to that shown before is observed: 78%, 67%, and 18% difference in parameters k_{σ}_{₁}_{→σ}_{₂}, k_{σ}_{₂}_{→σ}_{₁} and β₁ respectively. A ratio of likelihoods again favors an estimate

$$ln ( \frac{P ( {m}^{=} \vert {θ}^{′} )}{P ( {m}^{=} \vert θ )} ) \approx 4.7 \times 1 {0}^{3} .$$

This again suggests the presence of local maxima that may lead to incorrect parameter value estimates even when assuming a simpler model with fewer states. Once more, this result underscores the need for simultaneous optimization methods such as the methods outlined herein.
6.2.2 S. cerevisiae

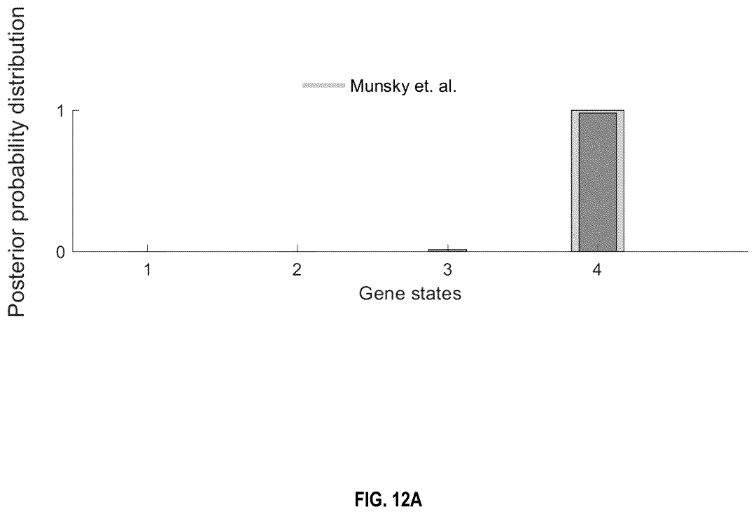

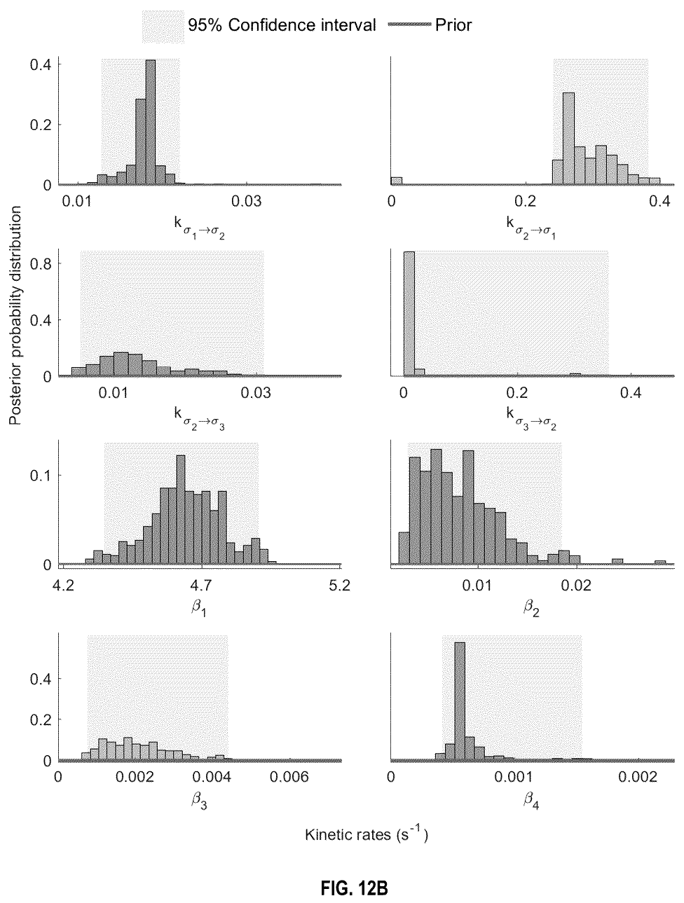

FIGS. 12A and 12B show results of model inference on the STL1 pathway in S. cerevisiae cells. This section compares findings on gene states and parameters inferred to estimates of gene parameters alone (and gene states posited) in other works. Analysis confirms the four states of chromatin reorganization of the STL1 gene in S. cerevisiae conjectured by previous methods prior to parameter estimation. However, as outlined in Section 2 and Section 3.2.1, this pre-specification of gene state numbers may lead to parameter estimates which only locally maximize the likelihood for a given set of observations. Owing to improved exploration of the space of models, the present method learns parameters (θ′, where posterior probability distributions for possible gene states are shown in FIG. 12A and posterior probability distributions for kinetic rate parameter values are shown in FIG. 12B) which are calculated to be more likely than those found by other methods by a factor of in

$$ln ( \frac{P ( {m}^{=} \vert {θ}^{′} )}{P ( {m}^{=} \vert θ )} ) \approx 350$$

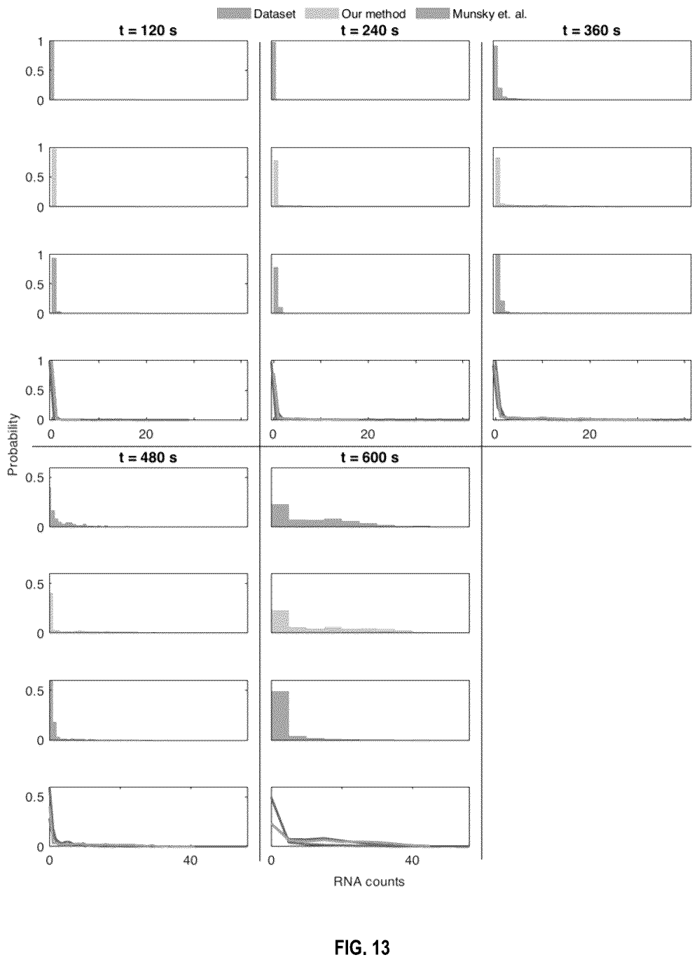

for STL1 transcription. See FIG. 13 for a comparison of predictive distributions of the type referred to in Section 6.2.1.

### 7. Discussion

Inferring the most probable regulatory network structure for a given set of observed snapshot RNA expression data presents unique challenges that have stood in the way of accurately identifying the number and connectivity of biophysical reactions and constituent parameters, whether separately or simultaneously. The present approach achieves model and parameter inference in a self-consistent and simultaneous fashion and improves upon limitations of other approaches, including 1) the assumption of steady-state dynamics and 2) separation of model selection of gene state numbers and parameter inference.

Effectiveness of the system applying the methods outlined herein are using both experimental and simulated snapshot RNA expression data. For E. coli in fast-growth media, the present system determined (FIG. 5) that a three-state model was more probable compared to the previously utilized two-state model. The additional state is an intermediate production state of the IacZ gene, with an intermediate rate of production lying between ‘on’ and ‘off’ rates assumed in a previous analysis. For the STL1 pathway in S. cerevisiae, the present system con-firmed that the previously utilized four-state network, with multiple production states, is the most likely model. Critically, the approach detailed here does not a priori assume the number or connectivity of gene states. Finally, the present disclosure demonstrates the robustness of the present methods using synthetic snapshot RNA expression data created by simulated regulatory networks designed to challenge any computational inference approach. These results demonstrate a general, simultaneous, self-consistent method to infer gene regulatory models and associated rates from snapshot RNA expression data obtained by smFISH.

Several additional extensions can be made to the system and associated methods outlined herein. First, with minimal modification, the present approach may utilize spatial information contained in snapshot RNA expression data quantified by smFISH to determine, for instance, transport rates of RNA from nucleus to cytoplasm. Indeed, the additional constraint of RNA transport from the nucleus to cytoplasm has previously improved parameter identification. Second, modifications of the measurement model within the present system may allow for time-varying rates of transcription, gene state transitions, and RNA degradation. Finally, as the density of gene species increases using highly multiplexed smFISH methods, the flexible network connectivity of the present approach may allow regulatory models to explore the most-likely regulatory networks for co-varying gene expression.

The above generalizations will introduce additional complexity to likelihood computation, which is already the costliest inference step. The additional complexity is directly due to the increase in the state number and complexity of the connectivity map. Both extensions would alter the generator matrix A (see Section 5.2), making it either larger for number of states or denser for connectivity. In the case where the generator matrix remains sparse, the CME solution's time cost scales roughly linearly with A's size, and FSP based Krylov subspace methods may be more optimal than the CME solution method used here. How the computational cost of A's CME scales with density is more complex than the number of states. Above a certain density, the recently proposed Quantized Tensor Train method may be more efficient, as the FSP based Krylov subspace approach uses incremental time stepping rather than jumping immediately to the times desired for analysis. Alternatively, there have been promising attempts to solve ODEs using neural networks. In addition to facilitating the difficulties arising due to dense CME generator matrices, neural network approaches may further enable parameter inference for non-Markovian models of gene transcription.

It may also be possible to deduce gene networks from direct image gene expression dynamics in real-time within living cells. However, such approaches obtain real-time kinetics of a limited number of molecules at the expense of higher data density. What is more, genetic manipulation limits the accessible insight to local molecular and biophysical interactions.

The desire to understand downstream consequences of gene expression, for example, through predictive modeling, directly motivates the use of snapshot RNA expression data especially for higher data density. While removing temporal correlations in snapshot RNA expression data immediately hinders the ability to obtain direct insight into gene regulatory dynamics, the knowledge gaps may be filled by increasing the density of time points, RNA species, cells, and stimuli conditions in snapshot RNA expression data. Indeed, the present disclosure shows how to maximize the information deduced from snapshot RNA expression data and obtain probabilities over gene regulatory network structures and constituent rates. This is achieved by reframing the gene regulatory network identification problem within the Bayesian nonparametric paradigm and developing the requisite tools for inference over gene states, connectivity, and parameters. The probabilistic output of the approach introduced may now allow us to learn networks reflecting the confidence that any given snapshot RNA expression data set supports.

It should be understood from the foregoing that, while particular embodiments have been illustrated and described, various modifications can be made thereto without departing from the spirit and scope of the invention as will be apparent to those skilled in the art. Such changes and modifications are within the scope and teachings of this invention as defined in the claims appended hereto.

What is claimed is:

1. A system, comprising:
a processor in communication with a memory, the memory including instructions executable by the processor to:
access observation data associated with a gene network including a quantity of RNA for a plurality of cells observed across a plurality of time points;
sample a set of probability values associated with observing the observation data for values of each respective parameter of a plurality of parameters through a measurement model, including:
a set of discrete parameters including one or more gene states observable within the observation data and a success probability of each respective gene state of the one or more gene states being active; and
a set of continuous parameters including kinetic rates associated with transitions between the one or more gene states based on a local geometry of the measurement model across the plurality of time points; and
jointly infer, based on the set of probability values and the observation data, a set of most probable values of the plurality of parameters.

2. The system of claim 1, the memory including instructions executable by the processor to:
apply a Gibbs sampling scheme to iteratively sample probability values associated with values of each respective model parameter of the gene network from the measurement model.

3. The system of claim 2, the memory including instructions executable by the processor to:
sample the success probability for a gene state of the one or more gene states using an adaptive Metropolis-Hastings sampling scheme encapsulated within the Gibbs sampling scheme.

4. The system of claim 2, the memory including instructions executable by the processor to:
iteratively sample probability values associated with the set of continuous parameters using a Hamiltonian Monte Carlo sampling scheme encapsulated within the Gibbs sampling scheme.

5. The system of claim 2, the memory including instructions executable by the processor to:
apply a Parallel Tempering scheme nested within the Gibbs sampling scheme for iteratively sampling probability values associated with values of each respective model parameter of the gene network from the measurement model.

6. The system of claim 1, the measurement model incorporating a nonparametric Bayesian formulation of a Chemical Master Equation that characterizes probabilistic temporal evolution of the gene network.

7. The system of claim 6, the nonparametric Bayesian formulation of the Chemical Master Equation incorporating a load vector that dynamically represents activity or inactivity of the one or more gene states observable within the observation data.

8. The system of claim 1, each respective gene state of the one or more gene states being represented as an element of a load vector, a value of the element of the load vector representing activity or inactivity of the associated gene state.

9. The system of claim 1, the measurement model including a joint posterior probability distribution expressive of a probability of observing the observation data given values of the plurality of parameters of the measurement model.

10. The system of claim 9, the posterior probability distribution being constructed based on a set of prior probability distributions associated with each respective parameter of the plurality of parameters of the measurement model.

11. The system of claim 10, each gene state of the one or more gene states being respectively expressed as an element of a load vector, wherein a prior probability distribution associated with the load vector includes a Beta-Bernoulli process prior probability distribution.

12. A method, comprising:
accessing observation data associated with a gene network including a quantity of RNA for a plurality of cells observed across a plurality of time points;
sampling a set of probability values associated with observing the observation data for values of each respective parameter of a plurality of parameters through a measurement model, including:
a set of discrete parameters including one or more gene states observable within the observation data and a success probability of each respective gene state of the one or more gene states being active; and
a set of continuous parameters including kinetic rates associated with transitions between the one or more gene states based on a local geometry of the measurement model across the plurality of time points; and
jointly inferring, based on the set of probability values and the observation data, a set of most probable values of the plurality of parameters.

13. The method of claim 12, further comprising:
applying a Gibbs sampling scheme to iteratively sample probability values associated with values of each respective model parameter of the gene network from the measurement model.

14. The method of claim 13, further comprising:
applying a Parallel Tempering scheme nested within the Gibbs sampling scheme for iteratively sampling probability values associated with values of each respective model parameter of the gene network from the measurement model.

15. The method of claim 13, further comprising:
sampling the success probability for a gene state of the one or more gene states using an adaptive Metropolis-Hastings sampling scheme encapsulated within the Gibbs sampling scheme.

16. The method of claim 13, further comprising:
iteratively sampling probability values associated with the set of continuous parameters using a Hamiltonian Monte Carlo sampling scheme encapsulated within the Gibbs sampling scheme.

17. The method of claim 12, the measurement model incorporating a nonparametric Bayesian formulation of a Chemical Master Equation that characterizes probabilistic temporal evolution of the gene network.

18. The method of claim 17, the nonparametric Bayesian formulation of the Chemical Master Equation incorporating a load vector that dynamically represents activity or inactivity of the one or more gene states observable within the observation data.

19. The method of claim 12, the measurement model including a joint posterior probability distribution expressive of a probability of observing the observation data given values of the plurality of parameters of the measurement model.

20. The method of claim 19, each gene state of the one or more gene states being respectively expressed as an element of a load vector, wherein a prior probability distribution associated with the load vector includes a Beta-Bernoulli process prior probability distribution.
